(19) United States
(12) Patent Application Publication
(10) Pub. No.: US 2022/0154184 A1
(43) Pub. Date: May 19, 2022

(54) INCREASING GENOME STABILITY AND REPROGRAMMING EFFICIENCY OF INDUCED PLURIPOTENT STEM CELLS

(71) Applicant: THE TRUSTEES OF COLUMBIA UNIVERSITY IN THE CITY OF NEW YORK, New York, NY (US)

(72) Inventors: Daniela Georgieva, New York, NY (US); Dietrich Egli, New York, NY (US)

(21) Appl. No.: 17/580,219

(22) Filed: Jan. 20, 2022

Related U.S. Application Data
(63) Continuation of application No. PCT/US2020/042978, filed on Jul. 22, 2020.
(60) Provisional application No. 62/877,052, filed on Jul. 22, 2019.

(51) Int. Cl. C12N 15/113; C12N 15/90; C12N 5/074; C12N 15/11

(52) U.S. Cl. CPC C12N 15/113; C12N 15/90; C12N 5/0696; C12N 15/111; C12N 2740/16043; C12N 2501/65; C12N 2506/1307; C12N 2510/00; C12N 2310/20

(57) ABSTRACT

The present disclosure concerns methods and compositions for generation of induced pluripotent stem cells with increased efficiency and genome stability. In particular aspects, induced pluripotent stem cells are generated from somatic cells following inhibition, reduction or downregulation of a particular protein or gene. In some embodiments, the protein is p53-binding protein or 53BP1.

### CROSS REFERENCE TO RELATED APPLICATION

The present application is a continuation of PCT Application No. PCT/US2020/042978, filed Jul. 22, 2020, which claims priority to U.S. Patent Application Ser. No. 62/877,052 filed Jul. 22, 2019, each of which is incorporated herein by reference in its entirety.

### FIELD

The current invention provides for methods and compositions for generating or producing induced pluripotent stem cells with improved genome stability, which leads to better derivation efficiency. The invention also provides for the cells produced by the method, including induced pluripotent stem cells and cells differentiated therefrom, that are suitable for transplantation or grafting into a subject for the prevention and/or treatment of disease, and useful for basic research and drug testing.

### BACKGROUND

Somatic cells can be reprogrammed to pluripotency upon ectopic expression of the four transcription factors OCT4, SOX2, KLF4 and cMYC (OSKM), which act as master regulators of the embryonic state (Takahashi and Yamanaka, 2006). The reprogrammed cell population, termed induced pluripotent stem (iPS) cells, is endowed with the capacity to proliferate indefinitely, but also to differentiate into any specialized cell type of the adult organism. These characteristics make iPS cells uniquely suitable for disease modelling and drug discovery in vitro, as well as, for the development of patient-specific cell replacement therapies. Nonetheless, overexpression of the reprogramming factors OSKM or OSK without cMYC results in increased levels of DNA damage (Gonzalez et al., 2013). Such increase is also seen during reprogramming by nuclear transfer, which does not entail the overexpression of transcription factors (Chia et al., 2017). These findings show that DNA damage is a general reprogramming phenomenon, is linked to replication stress (Ruiz et al., 2015) and results in de novo copy number alterations (Gore et al., 2011).

Genomic stability plays an important role in iPS cell generation as somatic cell reprogramming is severely compromised by mutations or knockdown of proteins implicated in DNA double-strand break (DSB) repair, including Brca1 and Brca2 (Gonzalez et al., 2013), CtIP (Gomez-Cabello et al., 2017), Rad51 (Gonzalez et al., 2013), FancC and FancA (Muller et al., 2012), FancD2 (Raya et al., 2009), and Atm (Kinoshita et al., 2011). In contrast, ablation of tumor suppressors p53 (Hong et al., 2009; Utikal et al., 2009b), p21 (Kawamura et al., 2009) and Rb (Kareta et al., 2015) results in more efficient iPS cell generation. These observations suggest that reprogramming and oncogenic transformation are governed by shared molecular processes that maintain genome stability and pose limits on somatic cell proliferation in response to DNA damage. However, the source of reprogramming-induced DNA damage, the type of damage and the mechanisms by which this damage is repaired remain poorly understood, as most factors that affect reprogramming efficiency, including BRCA1, exert multiple cellular functions. For example, Rad51 has a strand-exchange independent function in processing stalled replication forks (Mason et al., 2019), CtIP prevents resection of stalled forks by DNA2 (Przetocka et al., 2018) and BRCA2 inhibits stalled fork degradation by MRE11 (Schlacher et al., 2011).

Although BRCA1 has been implicated in many cellular processes, two aspects of its function are thought to be especially important for genome stability. First, BRCA1 is required for homology-directed repair (HDR), which repairs DSBs with high fidelity (Moynahan et al., 1999). BRCA1 promotes the HDR pathway at multiple stages, including an early commitment step in which the decision is made to repair a DSB either by HDR or by non-homologous end-joining (NHEJ). At this level, BRCA1 promotes HDR by facilitating DNA resection, a process that converts DSB ends into 3′ ssDNA overhangs, which are resistant to NHEJ and serve as key intermediates for HDR (Chen et al., 2018). Through molecular mechanisms that are still unclear, BRCA1 directs DSB repair to the HDR pathway by countering the activities of 53BP1, a protein that facilitates NHEJ by inhibiting DNA resection. Second, BRCA1 also functions in a distinct pathway that protects stalled DNA replication forks from nucleolytic degradation (Pathania et al., 2014; Schlacher et al., 2012). Interestingly, the HDR and stalled fork protection (SFP) activities of BRCA1 appear to be genetically separable, and abrogation of SFP alone is sufficient to elicit chromosomal instability in response to replication stress (Billing et al., 2018). Thus, HDR and SFP both contribute to the genome maintenance functions of BRCA1, and as such both may also be involved in BRCA1-mediated somatic cell reprogramming.

As shown herein, inhibiting or reducing p53-binding protein or 53BP1 increased BRCA1, allowing HDR to occur during programming which promotes genome stability, reprogramming efficiency, and quality of iPS cells.

### SUMMARY

Embodiments of the present disclosure encompass methods and compositions related to stem cell and tissue engineering. In particular embodiments, the present disclosure concerns methods and compositions that relate to production or generation of induced pluripotent stem cells and in certain embodiments these induced pluripotent stem cells are subject to conditions to produce differentiated cells, including in the form of tissue. The tissue comprising the induced pluripotent stem cells may be provided to an individual in need thereof, for example an individual with a wound or an individual with a medical condition, all of which cell or tissue repair would be beneficial.

In certain embodiments, induced pluripotent stem cells are generated by methods described herein where one or more types of adult somatic cells are provided and one or more agents that inhibit, reduce, knock down or down regulate 53BP1 expression are introduced into the somatic cells. Thus, one embodiment is a method of generating a human induced pluripotent stem cell, comprising: introducing one or more agents which inhibit, reduce, knock down or down regulate 53BP1 into a human somatic cell, and culturing the cell under conditions to generate a human induced pluripotent stem cell.

In some embodiments, the somatic cell is an epidermal cell, a fibroblast, other cells of epithelial origin (mammary, lung, intestinal), or blood cells.

In certain embodiments of the disclosure, somatic cells are obtained from an individual for subjecting to methods disclosed herein, although in some cases somatic cells are obtained commercially. The somatic cells that are subject to generation of induced pluripotent stem cells, or differentiated cells therefrom, may be delivered to an individual by any suitable means in the art. In some cases, the induced pluripotent stem cells, produced by methods disclosed herein, or differentiated cells therefrom, are delivered to the individual from which the original adult somatic cells were obtained. However, in certain embodiments the induced pluripotent stem cells are delivered to the individual other than the individual(s) from which the somatic cells were obtained. Thus, in some embodiments of the disclosure, the somatic cells are autologous to the individual and in some embodiments, the somatic cells are allogeneic to the individual.

Agents employed for inhibition, reduction, knock down or downregulation of 53BP1 may be of any kind so long as there is a detectable level of inhibition, reduction, knock down or downregulation of 53BP1 expression or so long as there is a noticeable effect of the somatic cell having pluripotent cell characteristics. In specific embodiments the agent is a nucleic acid, a polypeptide, a peptide, a small molecule, chemicals, endonucleases, or a mixture thereof. In cases where the agent is a nucleic acid, the agent may directly target the 53BP1 mRNA. In specific embodiments, the nucleic acid is antisense oligonucleotide, miRNA, siRNA, shRNA, gRNA and combinations thereof. Any nucleic acid that targets may be present on an expression vector, such as a lentiviral vector, a retroviral vector, an adenoviral vector, or a plasmid.

Further embodiments of the present disclosure are compositions for use in the disclosed methods. Such compositions include compositions and vectors comprising one or more agents that inhibits, reduces, knock downs or down regulates 53BP1.

In still a further embodiment of the disclosure there is provided an induced pluripotent stem cell produced or generated by the methods disclosed herein.

In further embodiments of the disclosure, induced pluripotent stem cells are subjected to conditions to produce a differentiated cell. The differentiated cell may be of any kind, and a skilled artisan recognizes how to tailor differentiation conditions to result in the desired differentiated cell. In specific cases, the differentiated cells are blood cells, pancreatic cells, endocrine cells, neurons, astrocytes, oligodendrocytes, retinal epithelial cells (RPE), epidermal cells, hair cells, keratinocytes, hepatocytes, intestinal epithelial cells, lung alveolar cells, hematopoietic cells, endothelial cells, cardiomyocytes, smooth muscle cells, skeletal muscle cells, cartilage cells, bone cells, renal cells, adipocytes, chondrocytes, and osteocytes Differentiation can also be induced by injecting cells into mice to induce differentiation through non-cell autonomous factors provided by the stromal tissue of the host mouse.

Thus, yet a further embodiment is a method of providing a differentiated cell population comprising: (a) obtaining a population of iPS cells produced or generated using the methods disclosed herein; and (b) culturing the cells in conditions effective to differentiate the iPS cells into a differentiated cell population. For example, in some aspects, step (b) culturing the cells comprises culturing the cells in a defined media comprising one or more growth factors. In further aspects, methods of the embodiments are further defined as methods of providing a differentiated cell population that comprises blood cells, pancreatic endocrine cells, pancreatic cells, endocrine cells, neurons, astrocytes, oligodendrocytes, retinal epithelial cells (RPE), epidermal cells, hair cells, keratinocytes, hepatocytes, intestinal epithelial cells, lung alveolar cells, hematopoietic cells, endothelial cells, cardiomyocytes, smooth muscle cells, skeletal muscle cells, cartilage cells, bone cells, renal cells, adipocytes, chondrocytes, and osteocytes. neuronal cells, mast cells, pancreatic beta cells, cardiomyocytes, or hepatocytes. In some aspects, cells for use according to the embodiments are mouse, rat or human cells.

In some embodiments of the disclosure, there is a method of repairing damaged tissue in or treating an individual in need thereof, comprising the steps of introducing an agent to somatic cells, wherein the agent inhibits, reduces, knock downs or down regulates expression of 53BP1 in the somatic cells and culturing the cells under conditions to generate or produce a human induced pluripotent stem cells; subjecting the induced pluripotent stem cells to conditions to produce differentiated cells; and delivering the differentiated cells to the individual in need thereof. In some embodiments, the somatic cells are autologous. In some embodiments, the somatic cells are allogenic. In certain cases, the differentiated cells are delivered to the individual in the form of tissue, such as skin tissue, muscle tissue, neurons, or blood, for example.

The current disclosure also includes kits.

### BRIEF DESCRIPTION OF THE FIGURES

For the purpose of illustrating the invention, there are depicted in drawings certain embodiments of the invention. However, the invention is not limited to the precise arrangements and instrumentalities of the embodiments depicted in the drawings.

FIGS. 1A-1F. Phosphoprotein interaction of Brca1 with Abraxas, Bach1 and CtIP is required for reprogramming. FIG. 1A shows a schematic of the proteins and residues involved in Brca1 BRCT phosphorecognition. AbraxasS404, Bach1S994 and CtIPS327 interact with Brca1S1598. The indicated serine to alanine substitutions prevent the formation of Brca1 complexes A, B and C. FIG. 1B is a graph of Rad51 foci immunofluorescence quantification in induced pluripotent stem (iPS) cell lines, homozygous mutant for the indicated genotypes. The cell lines were treated with 10 Gy IR. Data was collected from 3-4 cell lines per genotype and analyzed by one-way ANOVA. A=Abraxas^{S404A/S404A}, B=Bach^{1S994A/S994A}, C=CtIP^{S327A/S327A}. FIG. 1C shows the ratio of dual allele targeting in each genotype to dual allele targeting in the control in a CRISPR/Cas9 based HR assay with 3 or more iPS cell lines, except for Brca1^{tr/+}, where 2 cell lines were analyzed. Statistical analysis was performed with one-way ANOVA. The comparison between AACC and AABBCC as well as BBCC and AABBCC was performed with an unpaired two-tailed student's t-test. FIG. 1D shows the results of DNA fiber analysis in a fork stalling assay with hydroxyurea (HU). At least 150 DNA fibers were measured per genotype from 2-3 experimental replicates and analyzed by one-way ANOVA. FIG. 1E shows the quantification of alkaline phosphatase (AP) staining and reprogramming efficiency. The number of AP positive colonies is shown as a ratio to wild type. Data was collected from 4 replicates per genotype, consisting of 3 biological and 1 experimental, with the exception for AABBCC where 2 experimental replicates were available. The data was analyzed by one-way ANOVA. The comparison between BBCC and AABBBCC was performed with an unpaired, two-tailed student's t-test. FIG. 1F is a graph of the size of E13.5 embryos. Data was collected from 3-9 embryos per genotype. The difference between wt ctrl and AABBCC was evaluated with an unpaired two-tailed student's t-test. All ANOVA analyses used Sidak's multiple comparisons test. *p<0.05, **p<0.01 and ***p<0.001 and ****p<0.0001. Biological replicates are defined as cells from different embryos of the same genotype, while experimental replicates involve the use of cells from the same embryo in different experiments. Technical replicates were included in some experiments, but not considered in the statistical analysis.

FIGS. 2A-2J. Stalled fork protection (SFP) is dispensable for reprogramming. FIG. 2A shows a schematic of Bard1-mediated SFP. The Bard1^{K607A} point mutation prevents the recruitment of the Brca1/Bard1 heterodimer to reversed stalled replication forks, which makes them vulnerable to Mre11-dependent degradation. FIG. 2B is the results of DNA fiber analysis with hydroxyurea. Data was collected from 2 biological replicates per genotype (at least 200 fibers per genotype in total) and analyzed with a two-tailed student's t-test. FIG. 2C shows immunofluorescence quantification of phospho H2AX(S139) foci in uninfected E13.5 MEF from the indicated genotypes. Data was collected from 3 biological replicates per genotype (≥320 cells/genotype) and analyzed by one-way ANOVA. FIG. 2D shows the immunofluorescence quantification for double strand break (DSB) marker phospho H2AX (S139) for the indicated genotypes. Foci were counted on reprogramming day 5 in fibroblasts derived from 2-3 biological replicates (≥260 cells/genotype) and statistical analysis was performed with one-way ANOVA. FIG. 2E shows the immunofluorescence quantification of ssDNA marker phospho RPA(S33) on reprogramming day 5 of the indicated genotypes. Data was collected from 2-3 biological replicates (≥240 cells/genotype) per genotype and analyzed by one-way ANOVA. FIG. 2F shows the quantification of phospho RPA(S33) foci in uninfected E13.5 MEF in 3 biological replicates per genotype (at least 250 cells per genotype). Analysis was performed with one-way ANOVA. FIG. 2G shows the results of cell proliferation data analysis with CFSE dye. Arrested cells retain CFSE and are detectable as a bright peak by flow cytometry. Data was pooled from 2-3 biological replicates and analyzed by one-way ANOVA. FIG. 2H shows quantification of E13.5 embryo size with indicated genotype. Data was collected from 7-13 embryos per genotype, except for Bard1^{S563F/S563F} where 1 embryo was measured. Statistical significance was evaluated by one-way ANOVA. FIG. 2I shows a graph of the quantification of alkaline phosphatase (AP) staining and reprogramming efficiency for the indicated genotypes. The number of AP positive colonies is shown as a ratio to wild type. Data was collected from 4-6 replicates per genotype in total, consisting of 3 biological replicates and 1 to 3 experimental replicates. Analysis was performed with one-way ANOVA. FIG. 2J is quantification of alkaline phosphatase (AP) staining and reprogramming efficiency at day 20 relative to wild-type of Bard1^{S563F/+} and Bard1^{S563F/563F}. The number of AP positive colonies was first normalized to the number of plated cells and the efficiency of infection and then shown as a ratio to wild type. Data was collected from up to 7 replicates per genotype, consisting of 3 biological and up to 4 experimental replicates. Analysis was performed with one-way ANOVA. All ANOVA analyses used Sidak's multiple comparisons test. *p<0.05, **p<0.01 and ***p<0.001 and ****p<0.0001.

FIGS. 3A-3N. HDR-specific rescue of Brca1 function restores reprogramming. FIG. 3A shows a schematic of the strategy for rescuing SFP or HDR by ablation of Smarcal1 or 53bp1, respectively. FIG. 3B is the results of DNA fiber analysis in a fork stalling assay with hydroxyurea (HU). At least 120 DNA fibers were measured per genotype from 2-3 biological replicates and analyzed by one-way ANOVA. FIG. 3C show the results of DNA fiber analysis in a fork stalling assay with pyridostatin (PDS). Data was collected from 2-3 biological replicates (>180 fibers per genotype in total) and analyzed with one-way ANOVA. FIG. 3D shows the immunofluorescence quantification for double strand break (DSB) marker phospho H2AX(S139) for indicated genotypes. Foci were counted on reprogramming day 5 in fibroblasts derived from 2-3 biological replicates per genotype (≥410 cells/genotype). Statistical analysis was performed with one-way ANOVA. FIG. 3E shows the immunofluorescence quantification of phospho H2AX(S139) in uninfected E13.5 MEFs. Data was collected from 2-3 biological replicates (≥150 cells/genotype) per genotype and analyzed by one-way ANOVA. FIG. 3F shows immunofluorescence quantification of ssDNA marker phospho RPA(S33) on reprogramming day 5. Data was collected from 2-3 biological replicates (≥140 cells/genotype) per genotype and analyzed by one-way ANOVA. FIG. 3G shows results of a cell proliferation analysis with CFSE dye. Data was pooled from up to 5 replicates per genotype, consisting of 3 biological replicates and up to 2 experimental replicates. The exception was Brca1^{tr/tr}, where 2 biological replicates were analyzed. Statistical significance was determined with a 2-tailed, unpaired student's t-test. FIG. 3H is the results of an apoptosis analysis with Annexin V and propidium iodide (PI). Data was collected from 2-3 biological replicates per genotype and analyzed by one-way ANOVA. FIG. 3I is the quantification of E13.5 embryo size with the designated genotype. In each genotype, the area of 3-11 embryos was measured, except for Brca1^{tr/tr}, Smarcal1^{-/-} and Brca1^{tr/+}, for which 2 embryos per genotype were available. Statistical analysis was performed with one-way ANOVA. The Brca1^{tr/tr} and Brca1^{tr/tr}, 53bp1^{-/-} genotypes were compared with an unpaired, two-tailed student's t-test. FIG. 3J is the quantification of alkaline phosphatase (AP) staining and reprogramming efficiency for the indicated genotype. The number of AP positive colonies is shown as a ratio to wild type. Brca1 mutant cells were plated at 600-800 cells/mm² to ensure formation of colonies, while all other genotypes reprogramed well at 100-200 cells/mm². Data was collected from up to 12 replicates per genotype, consisting of 3 biological and up to 9 experimental replicates. The exception was Smarcal1^{-/-}, for which 2 biological replicates in total were available. Data analysis was performed with one-way ANOVA. FIG. 3K is quantification of flow cytometry analysis of HDR proficiency using a CRISPR/Cas9-based assay with 3 or more induced pluripotent stem (iPS) cells lines per genotype, except for Brca1^{tr/+}, where 2 cells lines were available. The data is shown as a ratio of dual allele targeting in each genotype to dual allele targeting in the control. Statistical analysis was performed with one-way ANOVA. The difference between wt ctrl and 53bp1−/− was evaluated with a 2-tailed, unpaired student's t-test. Data analysis was performed with one-way ANOVA. FIG. 3L is quantification of flow cytometry analysis of HDR proficiency with a CRISPR/Cas9-based assay of 53bp1−/− and wt ctrl. Data was collected from 5 replicates per genotype, consisting of 3 biological and 2 experimental replicates. Statistical significance was evaluated with an unpaired, two-tailed student's t-test. FIG. 3M is the quantification of AP staining and reprogramming efficiency. Data was collected from up to 7 replicates per genotype, consisting of 3 biological and up to 4 experimental replicates. The exception was Brca1^{tr/+}, Smarcal1^{-/-} for which 2 biological replicates were available. The comparison between Brca1^{tr/+} and Brca1^{tr/+}, 53bp1^{-/-} was performed with a two-tailed, unpaired student's t-test Statistical significance was determined with a 2-tailed, unpaired student's t-test. FIG. 3N is a Western blot for p21 and tubulin on protein, harvested from wild type and 53bp1 mutant MEFs after treatment with 8 Gy of IR. All ANOVA analyses used Sidak's multiple comparisons test. *p<0.05, **p<0.01 and ***p<0.001 and ****p<0.0001.

FIGS. 4A-4F. Replication-induced one-ended double strand breaks limit reprogramming. FIG. 4A shows the immunofluorescence quantification of 53bp1 foci of the indicated genotypes on reprogramming day 5. Data was collected from 3 biological replicates per genotype, except for the control where 2 biological replicates were available. For each genotype, at least 280 cells were analyzed and statistical significance was determined by one-way ANOVA. FIG. 4B shows the quantification of staining of 53bp1 foci in wild-type uninfected MEFs, treated with 0.2 uM aphidicolin for 3 days. At least 1000 cells were analyzed per condition and statistical significance was determined with an unpaired two-tailed student's t-test. FIG. 4C is a graph of genotype-specific sensitivity evaluation to treatment with 0.2 uM aphidicolin for 8 days during reprogramming. FIG. 4D is a graph of genotype-specific sensitivity evaluation to treatment with 10 nM topotecan for 8 days during reprogramming. FIG. 4E is a graph of genotype-specific sensitivity evaluation to a single dose of 6 Gy IR. For FIGS. 4C, 4D and 4E, data was collected from up to 9 replicates, consisting of 3 biological and up to 6 experimental replicates per genotype and analyzed by one-way ANOVA. The comparison between wt and 53bp1^{-/-} in FIG. 4D as well as the comparisons in FIG. 4E were performed with an unpaired, two-tailed student's t-test. FIG. 4F is quantification of AP staining and reprogramming efficiency for wild type MEFs treated with 5 uM DNA PK inhibitor for 8 days during reprogramming. Data was collected from 3 biological replicates and analyzed with an unpaired, two-tailed student's t-test. All ANOVA analyses used Sidak's multiple comparisons test. *p<0.05, **p<0.01 and ***p<0.001 and ****p<0.0001.

### DETAILED DESCRIPTION

The disclosure herein provides a novel way to generate induced pluripotent stem (iPS) cells from somatic cells using inhibition, reduction, knock down or down regulation of one gene, including 53BP1. These pluripotent stem cells can then be used to generate differentiated cells and tissues to repair damaged tissues or to treat a patient. Exemplary compositions include at least compositions comprising the agents, vectors comprising an inhibitory nucleic acid, induced pluripotent stem (iPS) cells generated using the methods disclosed herein, differentiated cell types derived from these induced pluripotent stem cells, and engineered tissues derived from these iPS cells.

### Definitions

The terms used in this specification generally have their ordinary meanings in the art, within the context of this invention and the specific context where each term is used. Certain terms are discussed below, or elsewhere in the specification, to provide additional guidance to the practitioner in describing the methods of the invention and how to use them. Moreover, it will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of the other synonyms. The use of examples anywhere in the specification, including examples of any terms discussed herein, is illustrative only, and in no way limits the scope and meaning of the invention or any exemplified term. Likewise, the invention is not limited to its preferred embodiments.

The term “inhibits”, “down regulates”, “knock down” and the like as used herein refers to reduction of expression of a gene product (RNA or protein).

As used herein, the term “induced pluripotent stem cells” commonly abbreviated as iPS cells or iPSCs, refers to a type of pluripotent stem cell artificially generated from a non-pluripotent cell, typically an adult somatic cell, or terminally differentiated cell, such as fibroblast, a hematopoietic cell, a myocyte, a neuron, an epidermal cell, or the like.

As used herein, the term “somatic cell” refers to any diploid cell forming the body of an organism.

As used herein, the term the terms “differentiation”, “cell differentiation” and the like refer to a process by which a less specialized cell (i.e., stem cell) develops or matures or differentiates to possess a more distinct form and/or function into a more specialized cell or differentiated cell, (i.e., pancreatic beta cell).

A cell that results from this process termed herein as a “differentiated cell” and can include pancreatic cells, endocrine cells, as well as neurons, astrocytes, oligodendrocytes, retinal epithelial cells (RPE), epidermal cells, hair cells, keratinocytes, hepatocytes, intestinal epithelial cells, lung alveolar cells, hematopoietic cells, endothelial cells, cardiomyocytes, smooth muscle cells, skeletal muscle cells, cartilage cells, bone cells, renal cells, adipocytes, chondrocytes, and osteocytes.

As used herein, the expressions “cell,” “cell line,” and “cell culture” are used interchangeably and all such designations include progeny. It is also understood that not all progeny will have precisely identical DNA content, due to deliberate or inadvertent mutations. Mutant progeny that have the same function or biological activity as screened for in the originally transformed cell are included. Where distinct designations are intended, it will be clear from the context.

With respect to cells, the term “isolated” refers to a cell that has been isolated from its natural environment (e.g., from a tissue or subject). The term “cell line” refers to a population of cells capable of continuous or prolonged growth and division in vitro. Often, cell lines are clonal populations derived from a single progenitor cell. It is further known in the art that spontaneous or induced changes can occur in karyotype during storage or transfer of such clonal populations. Therefore, cells derived from the cell line referred to may not be precisely identical to the ancestral cells or cultures, and the cell line referred to includes such variants. As used herein, the terms “recombinant cell” refers to a cell into which an exogenous DNA segment, such as DNA segment that leads to the transcription of a biologically-active polypeptide or production of a biologically active nucleic acid such as an RNA, has been introduced.

As used herein the specification, “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

In accordance with the present disclosure, there may be numerous tools and techniques within the skill of the art, such as those commonly used in molecular immunology, cellular immunology, pharmacology, and microbiology. See, e.g., Sambrook et al. (2001) Molecular Cloning: A Laboratory Manual. 3rd ed. Cold Spring Harbor Laboratory Press: Cold Spring Harbor, N.Y.; Ausubel et al. eds. (2005) Current Protocols in Molecular Biology. John Wiley and Sons, Inc.: Hoboken, N.J.; Bonifacino et al. eds. (2005) Current Protocols in Cell Biology. John Wiley and Sons, Inc.: Hoboken, N.J.; Coligan et al. eds. (2005) Current Protocols in Immunology, John Wiley and Sons, Inc.: Hoboken, N.J.; Coico et al. eds. (2005) Current Protocols in Microbiology, John Wiley and Sons, Inc.: Hoboken, N.J.; Coligan et al. eds. (2005) Current Protocols in Protein Science, John Wiley and Sons, Inc.: Hoboken, N.J.; and Enna et al. eds. (2005) Current Protocols in Pharmacology, John Wiley and Sons, Inc.: Hoboken, N.J.

### Abbreviations

iPS or iPSC—induced pluripotent stem (cell)
HDR—homology-directed repair
SFP—stalled fork protection
DSB—double strand break
NHEJ—non-homologous end-joining
wt ctrl—wild-type control
MEF—mouse embryonic fibroblast
IR—irradiation
53BP1—p53-binding protein

### Promoting Homologous Recombination Promote Genomic Stability in Somatic Cell

Reprogramming Reprogramming to pluripotency is associated with DNA damage and requires the functions of the homologous recombination protein BRCA1. Here, using the physical and genetic interactions of BRCA1 with its binding partner BARD1, the DNA translocase SMARCAL1, the non-homologous end joining (NHEJ) factor 53BP1 as well as phosphoprotein binding partners ABRAXAS, BACH1/BRIP1/FANCJ and CtIP, the relevance of stalled fork protection (SFP), stalled fork processing and homology directed repair (HDR) in somatic cell reprogramming was determined. Surprisingly, compromised stalled fork stability was inconsequential for the transition to pluripotency, while deficiency in HDR alone impaired reprogramming. Restoring HDR through inactivation of 53bp1 restored reprogramming in Brca1 mutants and increased the efficiency of iPS cell generation in wild-type fibroblasts. Furthermore, 53bp1 null cells had reduced sensitivity to replication fork stalling agents, aphidicolin and topotecan, during reprogramming but remained vulnerable to irradiation which generates two-ended double strand breaks. These results demonstrated that replication-induced one-ended double strand breaks, which require repair by homologous recombination, are the primary limitation to somatic cell reprogramming.

Brca1^{tr/tr} Smarcal1^{-/-} cells are deficient specifically for the HDR, but not the SFP function of Brca1 and yet reprogram poorly. The genetic interaction of 53BP1 with BRCA1 is known to regulate the balance of DNA repair pathway choice at two-ended double strand breaks. Together with RIF1, 53BP1 acts in a competitive and antagonistic manner to BRCA1-CtIP to inhibit break end resection (Escribano-Diaz et al., 2013). Inactivation of 53bp1 in Brca1 mutant cells rescued their HDR competence, in line with previous reports (Bunting et al., 2010). Loss of 53bp1 reduced the amount of replication stress, DNA damage, apoptosis and fully rescued the efficiency of iPS cell generation in the absence of Brca1.

Surprisingly, improved reprogramming upon disruption of 53bp1 was noted in two additional genotypes: 53bp1 deficiency in otherwise wild-type cells, as well as, in heterozygous Brca1^{tr/+} mutants. This result challenges a previous study which reported a 2-fold reduction in iPS cells generation for 53bp1^{-/-} mouse fibroblasts compared to controls (Marión et al., 2009). The strength of the conclusion herein comes from examining the consequences of 53bp1 loss in three different genotypes: wild-type, Brca1^{tr/+} and Brca1^{tr/tr}, all of which reprogram better in the absence of 53bp1. Since BRCA1 and 53BP1 compete to determine repair pathway choice, the absence of 53bp1 in wild-type cells enhances the efficiency of HDR as it is shown in an HDR assay.

In addition to its role in DSB repair by non-homologous end joining, 53BP1 has a separate function in the stimulation of p53-dependent transcription. Loss of 53BP1 has been reported to impair the induction of p21, as well as, pro-apoptotic targets BAX and PUMA/BBC3 in human metastatic adenocarcinoma cells (Cuella-Martin et al., 2016). Loss of p53 or downregulation of p21 improves iPS cell generation, providing an alternative mechanism by which ablation of 53bp1 could increase reprogramming. However, another study showed normal stabilization of p53 in 53bp1^{-/-} mouse thymocytes and upregulation of p21 in response to IR (Ward et al., 2005). In the experimental system-mouse embryonic fibroblasts used herein, no changes in proliferation or apoptosis in 53bp1^{-/-} cells were noted and normal induction of p21 was found in 53bp1 mutant cells in response to treatment with ionizing irradiation.

Therefore, the improvement in reprogramming efficiency in the absence of 53bp1 is not due to compromised induction of p53 targets. In further support of this notion, the increase of reprogramming efficiency in 53bp1 mutant cells is specific to one-ended DSBs, and not seen upon induction of two-ended DSBs as would be expected from a deficiency in p53, which causes resistance to irradiation.

To determine the specific type of DNA damage limiting to reprogramming, a series of experiments was conducted, which used drug treatment to increase the load of one-ended DSBs or irradiation to induce the accumulation of two-ended DSBs. It was noted that 53bp1^{-/-} fibroblasts were less sensitive to inducers of one-ended DSBs during reprogramming but remained more vulnerable to two-ended DSBs as previously reported for 53bp1^{-/-} mice and embryonic cells. Importantly, loss of 53bp1 in Brca1^{tr/tr} cells reduced their sensitivity to both aphidicolin and topotecan-induced one-ended DSBs but resulted in no improvement when IR was administered. These observations collectively indicated that the limiting factor for reprogramming to pluripotency is a one-ended double strand break which requires homology-directed repair.

In contrast to the deletions of p21, Rb or p53 which accelerate tumor formation and improve reprogramming efficiency, the phenotype of BRCA1 loss has opposite effects: it increases tumorigenesis but impairs reprogramming. Both phenotypes are specifically due to the HDR function of BRCA1. Mice with HDR deficiency are tumor prone, while SFP loss has no effect on tumor formation (Billing et al., 2018) (Table 2). In the system used herein, it was shown that the HDR, but not the SFP function of BRCA1 is required for somatic cell reprogramming. Loss of 53bp1 restores HDR and reduces the tumor incidence in Brca1^{tr/tr} animals (Cao et al., 2009), and also rescues reprogramming (Table 2). However, some BRCA1-deficient cancers inactivate 53BP1 (Jaspers et al., 2013) which can reduce sensitivity to treatment, suggesting that the effect of HDR deficiency in cancer may depend on the stage of tumorigenesis. This BRCA1 paradox, where HDR is required for tumor suppression, but also enables tumor growth and reprogramming, remains unresolved. A novel perspective is provided here: the one-ended DSB acts as primary inhibitor to abnormal cell type transitions by initiating a signaling cascade which prevents proliferation and stabilizes the differentiated state (Sui et al., 2020). The balance of DNA repair pathways in this context protects genome integrity.

Agents with Inhibit, Reduce, Knock down or Down Regulate 53BP1

A wide variety of suitable agents may be employed, guided by art-recognized criteria such as efficacy, toxicity, stability, specificity, half-life, etc. for the inhibition, reduction, knock down or down regulation of 53BP1. Furthermore, the mechanism of inhibition may be at the genetic level (e.g., interference with or inhibit expression, transcription or translation, etc.) or at the protein level (e.g., binding, competition, etc.).

In some embodiments, the agent which inhibits, reduces, knocks down or down regulated 53BP1, i.e., inhibitory nucleic acid, is present on the same vector as the other reprogramming factors, e.g., OSKM factors. In some embodiments, the agent which inhibits, reduces, knocks down or down regulates 53BP1 is present on a separate vector as the other reprogramming factors. In some embodiments, the agent is contacted or incubated with the cell by culturing the cells in media comprising the agent(s).

Small Molecule Inhibitors

As used herein, the term “small molecules” encompasses molecules other than proteins or nucleic acids without strict regard to size. Non-limiting examples of small molecules that may be used according to the methods and compositions of the present disclosure include, small organic molecules, peptide-like molecules, peptidomimetics, carbohydrates, lipids or other organic (carbon containing) or inorganic molecules.

Inhibitory Proteins

In certain embodiment, the agent used in the present methods and composition is a protein or peptide that inhibits the activity of the 53BP1 protein. One such protein has been identified and is a ubiquitin variant. See Canny, et al. 2018.

Inhibitory Nucleic Acids

In certain embodiments, the agent used in the present methods and compositions is a polynucleotide that reduces expression of 53BP1. Thus, the method involves introducing a polynucleotide that specifically targets nucleotide sequence(s) encoding 53BP1. The polynucleotides reduce expression of 53BP1, to yield reduced levels of the gene product (the translated polypeptide). The nucleic acid target of the polynucleotides may be any location within the gene or transcript of 53BP1. The sequence for 53BP1 can be found on the National Center for Biotechnology Information Database (Gene ID: 7158) and can be used to design polynucleotide that reduces expression of 53BP1 in conjunction with computer programs.

Any number of means for inhibiting 53BP1 activity or gene expression can be used in the disclosed methods. For example, a nucleic acid molecule complementary to at least a portion of a 53BP1 encoding nucleic acid can be used to inhibit 53bp1 gene expression.

RNA interference (RNAi) is a biological process where RNA molecules are used to inhibit gene expression. Typically, short RNA molecules are created that are complementary to endogenous mRNA and when introduced into cells, bind to the target mRNA. Binding of the short RNA molecule to the target mRNA functionally inactivates the target mRNA and sometimes leads to degradation of the target mRNA.

Historically, two types of short RNA molecules have been used in RNAi applications. Small interfering RNA (siRNA) are typically double-stranded RNA molecules, 20-25 nucleotides in length. When transfected into cells, siRNA inhibit the target mRNA transiently until they are also degraded within the cell. Other means for inhibiting gene expression using short RNA molecules, for example, include small temporal RNAs (stRNAs), and micro-RNAs (miRNAs).

Small hairpin RNAs (shRNA) are sequences of RNA, typically about 80 base pairs in length, that include a region of internal hybridization that creates a hairpin structure. shRNA molecules are processed within the cell to form siRNA which in turn knock down gene expression. The benefit of shRNA is that they can be incorporated into plasmid vectors and integrated into genomic DNA for longer-term or stable expression, and thus longer knockdown of the target mRNA.

Short interfering RNAs silence genes through an mRNA degradation pathway, while stRNAs and miRNAs are approximately 21 or 22 nt RNAs that are processed from endogenously encoded hairpin-structured precursors, and function to silence genes via translational repression. See, e.g., McManus et al. (2002). RNA 8(6):842-50; Morris et al. (2004). Science 305(5688):1289-92; He and Hannon. (2004). Nat. Rev. Genet. 5(7):522-31.

MicroRNA can also be used to inhibit 52BP1. MicroRNAs are small non-coding RNAs averaging 22 nucleotides that regulate the expression of their target mRNA transcripts by binding. Binding of microRNAs to their targets is specified by complementary base pairing between positions 2-8 of the microRNA and the target 3′ untranslated region (3′ UTR), an mRNA component that influences translation, stability and localization. Such microRNA can be designed using the known sequence of the 3′UTR of 53bp1. Additionally, this microRNA can also be modified for increasing other desirable properties, such as increased stability, decreased degradation in the body, and increased cellular uptake.

Alternatively, double-stranded (ds) RNA is a powerful way of interfering with gene expression in a range of organisms that has recently been shown to be successful in mammals (Wianny and Zernicka-Goetz. (2002), Nat. Cell. Biol. 2:70-75). Double stranded RNA corresponding to the sequences of a 53BP1 polynucleotide can be introduced into cells.

The inhibitory nucleic acids may be an antisense nucleic acid sequence that is complementary to a target region within the mRNA of 53BP1. The antisense polynucleotide may bind to the target region and inhibit translation. The antisense oligonucleotide may be DNA or RNA or comprise synthetic analogs of ribo-deoxynucleotides. Thus, the antisense oligonucleotide inhibits expression of 53BP1.

An antisense oligonucleotide can be, for example, about 7, 10, 15, 20, 25, 30, 35, 40, 45, 50, 55, 60, 65, 70, 75, 80, or more nucleotides in length.

Endonucleases

Methods for modification of genomic DNA are well known in the art. The term “DNA digesting agent” refers to an agent that is capable of cleaving bonds (i.e. phosphodiester bonds) between the nucleotide subunits of nucleic acids.

In one embodiment, the DNA digesting agent is a nuclease. Nucleases are enzymes that hydrolyze nucleic acids. Nucleases may be classified as endonucleases or exonucleases. An endonuclease is any of a group of enzymes that catalyze the hydrolysis of bonds between nucleic acids in the interior of a DNA or RNA molecule. An exonuclease is any of a group of enzymes that catalyze the hydrolysis of single nucleotides from the end of a DNA or RNA chain. Nucleases may also be classified based on whether they specifically digest DNA or RNA. A nuclease that specifically catalyzes the hydrolysis of DNA may be referred to as a deoxyribonuclease or DNase, whereas a nuclease that specifically catalyses the hydrolysis of RNA may be referred to as a ribonuclease or an RNase. Some nucleases are specific to either single-stranded or double-stranded nucleic acid sequences. Some enzymes have both exonuclease and endonuclease properties. In addition, some enzymes are able to digest both DNA and RNA sequences. 53BP1 may be inhibited by using a sequence-specific endonuclease that target the gene encoding 53BP1.

Non-limiting examples of the endonucleases include a zinc finger nuclease (ZFN), a ZFN dimer, a ZFNickase, a transcription activator-like effector nuclease (TALEN), or a RNA-guided DNA endonuclease (e.g., CRISPR/Cas9). Meganucleases are endonucleases characterized by their capacity to recognize and cut large DNA sequences (12 base pairs or greater). Any suitable meganuclease may be used in the present methods to create double-strand breaks in the host genome, including endonucleases in the LAGLIDADG and PI-Sce family.

One example of a sequence-specific nuclease system that can be used with the methods and compositions described herein includes the CRISPR system (Wiedenheft, et al. Nature, 482, 331-338 (2012); Jinek, et al. Science, 337, 816-821 (2012); Mali, et al. Science, 339, 823-826 (2013); Cong, et al. Science, 339, 819-823 (2013)). The CRISPR (Clustered Regularly interspaced Short Palindromic Repeats) system exploits RNA-guided DNA-binding and sequence-specific cleavage of target DNA. The guide RNA/Cas combination confers site specificity to the nuclease. A single guide RNA (sgRNA) contains about 20 nucleotides that are complementary to a target genomic DNA sequence upstream of a genomic PAM (protospacer adjacent motifs) site (NGG) and a constant RNA scaffold region. The Cas (CRISPR-associated) protein binds to the sgRNA and the target DNA to which the sgRNA binds and introduces a double-strand break in a defined location upstream of the PAM site. Cas9 harbors two independent nuclease domains homologous to HNH and RuvC endonucleases, and by mutating either of the two domains, the Cas9 protein can be converted to a nickase that introduces single-strand breaks (Cong, et al. Science, 339, 819-823 (2013)). It is specifically contemplated that the methods and compositions of the present disclosure can be used with the single- or double-strand-inducing version of Cas9, as well as with other RNA-guided DNA nucleases, such as other bacterial Cas9-like systems. The sequence-specific nuclease of the present methods and compositions described herein can be engineered, chimeric, or isolated from an organism. The nuclease can be introduced into the cell in form of a DNA, mRNA and protein. The applications of the CRISPR/Cas system to inhibiting or downregulating 53bp1 are easily adapted.

In one embodiment, the DNA digesting agent can be a site-specific nuclease. In another embodiment, the site-specific nuclease may be a Cas-family nuclease. In a more specific embodiment, the Cas nuclease may be a Cas9 nuclease.

In one embodiment, Cas protein may be a functional derivative of a naturally occurring Cas protein.

In addition to well characterized CRISPR-Cas system, a new CRISPR enzyme, called Cpf1 (Cas protein 1 of PreFran subtype) has recently been described. Cpf1 is a single RNA-guided endonuclease that lacks tracrRNA and utilizes a T-rich protospacer-adjacent motif. The authors demonstrated that Cpf1 mediates strong DNA interference with characteristics distinct from those of Cas9. Thus, in one embodiment of the present disclosure, CRISPR-Cpf1 system can be used to cleave a desired region within the targeted gene.

In further embodiment, the DNA digesting agent is a transcription activator-like effector nuclease (TALEN). TALENs are composed of a TAL effector domain that binds to a specific nucleotide sequence and an endonuclease domain that catalyzes a double strand break at the target site (PCT Patent Publication No. WO2011072246; Miller et al., Nat. Biotechnol. 29, 143-148 (2011); Cermak et al., Nucleic Acid Res. 39, e82 (2011)). Sequence-specific endonucleases may be modular in nature, and DNA binding specificity is obtained by arranging one or more modules. Bibikova et al., Mol. Cell. Biol. 21, 289-297 (2001). Boch et al., Science 326, 1509-1512 (2009).

ZFNs can be composed of two or more (e.g., 2-8, 3-6, 6-8, or more) sequence-specific DNA binding domains (e.g., zinc finger domains) fused to an effector endonuclease domain (e.g., the FokI endonuclease). Porteus et al., Nat. Biotechnol. 23, 967-973 (2005). Kim, et al. Proceedings of the National Academy of Sciences of USA, 93: 1156-1160 (2007); U.S. Pat. No. 6,824,978; PCT Publication Nos. WO1995/09233 and WO1994018313.

In one embodiment, the DNA digesting agent is a site-specific nuclease of the group or selected from the group consisting of omega, zinc finger, TALE, and CRISPR/Cas.

The sequence-specific endonuclease of the methods and compositions described here can be engineered, chimeric, or isolated from an organism. Endonucleases can be engineered to recognize a specific DNA sequence, by, e.g., mutagenesis. Seligman et al., Nucleic Acids Research_30:3870-3879 (2002). Combinatorial assembly is a method where protein subunits form different enzymes can be associated or fused. Arnould et al., Journal of Molecular Biology 355: 443-458 (2006). In certain embodiments, these two approaches, mutagenesis and combinatorial assembly, can be combined to produce an engineered endonuclease with desired DNA recognition sequence.

The sequence-specific nuclease can be introduced into the cell in the form of a protein or in the form of a nucleic acid encoding the sequence-specific nuclease, such as an mRNA or a cDNA. Nucleic acids can be delivered as part of a larger construct, such as a plasmid or viral vector, or directly, e.g., by electroporation, lipid vesicles, viral transporters, microinjection, and biolistics. Similarly, the construct containing the one or more transgenes can be delivered by any method appropriate for introducing nucleic acids into a cell.

Single guide RNA(s) used in the methods of the present disclosure can be designed so that they direct binding of the Cas-sgRNA complexes to pre-determined cleavage sites in a genome. In one embodiment, the cleavage sites may be chosen so as to release a fragment or sequence that contains a region of autosomal dominant disease-related gene.

sgRNA(s) used in the present disclosure can be between about 5 and 100 nucleotides long, or longer (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 60, 61, 62, 63, 63, 64, 65, 66, 67, 68, 69, 70, 71, 72, 73, 74, 75, 76, 77, 78, 79, 80, 81, 82, 83, 84, 85, 86, 87, 88, 89, 90, 91 92, 93, 94, 95, 96, 97, 98, 99, or 100 nucleotides in length, or longer). In one embodiment, sgRNA(s) can be between about 15 and about 30 nucleotides in length (e.g., about 15-29, 15-26, 15-25; 16-30, 16-29, 16-26, 16-25; or about 18-30, 18-29, 18-26, or 18-25 nucleotides in length).

To facilitate sgRNA design, many computational tools have been developed

Ribozyme

The inhibitor may be a ribozyme that inhibits expression of the 53BP1 gene.

Ribozymes can be chemically synthesized and structurally modified to increase their stability and catalytic activity using methods known in the art. Ribozyme encoding nucleotide sequences can be introduced into host cells through gene-delivery mechanisms known in the art.

### Nucleic Acid-Based Expression Systems

In some embodiments, there is a nucleic acid-based agent that targets 53BP1. In specific embodiments, the nucleic acid agent is present on a vector for expression in a eukaryotic cell.

Vectors

The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques (see, for example, Maniatis et al., 1988 and Ausubel et al., 1994).

The term “expression vector” refers to any type of genetic construct comprising a nucleic acid coding for a RNA capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host cell. In addition to control sequences that govern transcription and translation, vectors and expression vectors may contain nucleic acid sequences that serve other functions as well and are described infra.

Vectors of the present disclosure can comprise any of a number of promoters known to the art, wherein the promoter is constitutive, regulatable or inducible, cell type specific, tissue-specific, or species specific. In addition to the sequence sufficient to direct transcription, a promoter sequence of the invention can also include sequences of other regulatory elements that are involved in modulating transcription (e.g., enhancers, kozak sequences and introns). Many promoter/regulatory sequences useful for driving constitutive expression of a gene are available in the art and include, but are not limited to, for example, CMV (cytomegalovirus promoter), EF1a (human elongation factor 1 alpha promoter), SV40 (simian vacuolating virus 40 promoter), PGK (mammalian phosphoglycerate kinase promoter), Ubc (human ubiquitin C promoter), human beta-actin promoter, rodent beta-actin promoter, CBh (chicken beta-actin promoter), CAG (hybrid promoter contains CMV enhancer, chicken beta actin promoter, and rabbit beta-globin splice acceptor), TRE (Tetracycline response element promoter), H1 (human polymerase III RNA promoter), U6 (human U6 small nuclear promoter), and the like. Moreover, inducible and tissue specific expression of an RNA, transmembrane proteins, or other proteins can be accomplished by placing the nucleic acid encoding such a molecule under the control of an inducible or tissue specific promoter/regulatory sequence. Examples of tissue specific or inducible promoter/regulatory sequences which are useful for this purpose include, but are not limited to, the rhodopsin promoter, the MMTV LTR inducible promoter, the SV40 late enhancer/promoter, synapsin 1 promoter, ET hepatocyte promoter, GS glutamine synthase promoter and many others. Various commercially available ubiquitous as well as tissue-specific promoters can be found at http://www.invivogen.com/prom-a-list and https://www.addgene.org/. In addition, promoters which are well known in the art can be induced in response to inducing agents such as metals, glucocorticoids, tetracycline, hormones, and the like, are also contemplated for use with the invention. Thus, it will be appreciated that the present disclosure includes the use of any promoter/regulatory sequence known in the art that is capable of driving expression of the desired protein operably linked thereto.

In addition to promoters and enhancers, vectors can further comprise a specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments, the use of internal ribosome entry sites (IRES) elements are used to create multigene, or polycistronic, messages.

Vectors also comprise termination signals, polyadenylation signals, and origins of replication.

It is within the skill of the art for one to construct a vector into which a nucleic acid sequence can be inserted for introduction into a cell where it can be replicated

Plasmid Vectors

In certain embodiments, a plasmid vector is contemplated for use to transform a host cell. In general, plasmid vectors containing replicon and control sequences which are derived from species compatible with the host cell are used in connection with these hosts. The vector ordinarily carries a replication site, as well as marking sequences which are capable of providing phenotypic selection in transformed cells.

In addition, phage vectors containing replicon and control sequences that are compatible with the host microorganism can be used as transforming vectors in connection with these hosts.

Further useful plasmid vectors include pIN vectors; and pGEX vectors, for use in generating glutathione S-transferase (GST) soluble fusion proteins for later purification and separation or cleavage. Other suitable fusion proteins are those with beta-galactosidase, ubiquitin, and the like.

Viral Vectors

The ability of certain viruses to infect cells or enter cells via receptor-mediated endocytosis, and to integrate into host cell genome and express viral genes stably and efficiently have made them attractive candidates for the transfer of foreign nucleic acids into cells (e.g., mammalian cells). Components described herein may be a viral vector that target 53BP1. Non-limiting examples of virus vectors that may be used to deliver a nucleic acid are described below.

Adenoviral Vectors

A particular method for delivery of the nucleic acid involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and (b) to ultimately express a tissue or cell-specific construct that has been cloned therein. Knowledge of the genetic organization or adenovirus, a 36 kb, linear, double-stranded DNA virus, allows substitution of large pieces of adenoviral DNA with foreign sequences up to 7 kb.

AAV Vectors

The nucleic acid may be introduced into the cell using adenovirus assisted transfection. Increased transfection efficiencies have been reported in cell systems using adenovirus coupled systems. Adeno-associated virus (AAV) is an attractive vector system for use in the compositions of the present disclosure as it has a high frequency of integration and it can infect nondividing cells, thus making it useful for delivery of genes into mammalian cells, for example, in tissue culture or in vivo. AAV has a broad host range for infectivity. Details concerning the generation and use of rAAV vectors are described in U.S. Pat. Nos. 5,139,941 and 4,797,368, each incorporated herein by reference.

Retroviral Vectors

Retroviruses are useful as delivery vectors because of their ability to integrate their genes into the host genome, transferring a large amount of foreign genetic material, infecting a broad spectrum of species and cell types and of being packaged in special cell-lines.

In order to construct a retroviral vector, a nucleic acid (e.g., one comprising a targeting nucleic acid of interest) is inserted into the viral genome in the place of certain viral sequences to produce a virus that is replication-defective. In order to produce virions, a packaging cell line containing the gag, pol, and env genes but without the LTR and packaging components is constructed. When a recombinant plasmid containing a cDNA, together with the retroviral LTR and packaging sequences is introduced into a special cell line (e.g., by calcium phosphate precipitation for example), the packaging sequence allows the RNA transcript of the recombinant plasmid to be packaged into viral particles, which are then secreted into the culture media. The media containing the recombinant retroviruses is then collected, optionally concentrated, and used for gene transfer.

Lentiviruses are complex retroviruses, which, in addition to the common retroviral genes gag, pol, and env, contain other genes with regulatory or structural function. Lentiviral vectors are well known in the art. Some examples of lentivirus include the Human Immunodeficiency Viruses: HIV-1, HIV-2 and the Simian Immunodeficiency Virus: SIV. Lentiviral vectors have been generated by multiply attenuating the HIV virulence genes, for example, the genes env, vif, vpr, vpu and nef are deleted making the vector biologically safe.

Recombinant lentiviral vectors are capable of infecting non-dividing cells and can be used for both in vivo and ex vivo gene transfer and expression of nucleic acid sequences. For example, recombinant lentivirus capable of infecting a non-dividing cell wherein a suitable host cell is transfected with two or more vectors carrying the packaging functions, namely gag, pol and env, as well as rev and tat is described in U.S. Pat. No. 5,994,136. One may target the recombinant virus by linkage of the envelope protein with an antibody or a particular ligand for targeting to a receptor of a particular cell-type. By inserting a sequence (including a regulatory region) of interest into the viral vector, along with another gene which encodes the ligand for a receptor on a specific target cell, for example, the vector is now target-specific.

Other Viral Vectors

Other viral vectors may be employed in the current methods. Vectors derived from viruses such as vaccinia virus, sindbis virus, cytomegalovirus and herpes simplex virus may be employed.

Extra-Chromosomal Vectors

In certain embodiments, the disclosed methods make use of extra-chromosomal genetic elements (e.g., for expression of zinc finger nucleases or inhibitory nucleic acid targeted to the 0.53BP1 gene). For example, extra-chromosomally replicating vectors, or vectors capable of replicating episomally can be employed. In further aspects, RNA molecules (e.g., mRNAs, shRNAs, siRNAs, or miRNAs) can be employed.

A number of DNA viruses, such as adenoviruses, Simian vacuolating virus 40 (SV40), bovine papilloma virus (BPV), or budding yeast ARS (Autonomously Replicating Sequences)-containing plasmids also replicate extra-chromosomally in mammalian cells. These episomal plasmids are intrinsically free from all these disadvantages associated with integrating vectors. A lymphotrophic herpes virus-based system including Epstein Barr Virus (EBV) may also replicate extra-chromosomally and help deliver genetic elements to somatic cells. For example, episomal vector-based approaches may employ elements of an EBV-based system. The useful EBV elements are OriP and EBNA-1, or their variants or functional equivalents.

An additional advantage of systems based on extra-chromosomal vectors is that these exogenous elements can be lost with time after being introduced into cells, leading to self-sustained iPS cells or cells differentiated from iPS cells that are essentially free of the original elements.

### Vector Delivery and Cell Transformation

Suitable methods for nucleic acid delivery for transformation of an organelle, a cell, a tissue or an organism for use in the disclosed methods include virtually any method by which a nucleic acid (e.g., DNA) can be introduced into an organelle, a cell, a tissue or an organism, as described herein or as would be known to one of ordinary skill in the art. Such methods include, but are not limited to, direct delivery of DNA such as by ex vivo transfection, injection, including microinjection, electroporation, calcium phosphate precipitation, DEAE-dextran followed by polyethylene glycol, direct sonic loading, liposome mediated transfection, receptor-mediated transfection, microprojectile bombardment, agitation with silicon carbide fibers, Agrobacterium-mediated transformation, PEG-mediated transformation of protoplasts, desiccation/inhibition-mediated DNA uptake, and combinations thereof. Through the application of techniques such as these, organelle(s), cell(s), tissue(s) or organism(s) may be stably or transiently transformed.

### Methods of Generating Induced Pluripotent Stem Cells

The current method is to generate induced pluripotent stem cells more efficiently and which are more stable, by the inhibition, reduction, knock down or downregulation of 53BP1.

A human somatic cell is the starting material of the disclosed methods. The human somatic cell can be autologous to an individual or allogeneic to an individual.

In certain embodiments, induced pluripotent stem cells are generated by methods described herein where one or more types of adult somatic cells are provided and one or more agents that inhibit, reduce, knock down or down regulate 53BP1 expression are introduced into the somatic cells. Thus, one embodiment, is a method of generating a human induced pluripotent stem cell, comprising: introducing one or more agents which inhibit, reduce, knock down or down regulate 53BP1 into a human somatic cell, and culturing the cell under conditions to generate a human induced pluripotent stem cell.

In specific embodiments the agent is a nucleic acid, a polypeptide, a peptide, a small molecule, chemicals, endonucleases, or a mixture thereof. In cases where the agent is a nucleic acid, the agent may directly target the 53BP1 mRNA. In specific embodiments, the nucleic acid is antisense oligonucleotide, miRNA, siRNA, shRNA, gRNA and combinations thereof. Any nucleic acid that targets may be present on an expression vector, such as a lentiviral vector, a retroviral vector, an adenoviral vector, or a plasmid.

In some embodiments, the agent which inhibits, reduces, knocks down or down regulated 53BP1, i.e., inhibitory nucleic acid, is present on the same vector as the other reprogramming factors, e.g., OSKM factors. In some embodiments, the agent which inhibits, reduces, knocks down or down regulated 53BP1 is present on a separate vector as the other reprogramming factors. In some embodiments, the agent is introduced to the cell by culturing the cells in media comprising the agent(s).

In some embodiments, the agent which inhibits, reduces, knocks down or down regulated 53BP1 is introduced to the cell at the same time as the other reprogramming factors, i.e., the agent is introduced to the somatic cell at the beginning of the reprogramming process and is present to the end of the process.

In some embodiments, the agents which inhibits, reduces, knocks down or down regulated 53BP1 is transient, i.e., 53BP1 function returns to the iPSC after reprogramming.

A method of generating iPSC with improved genome stability is exemplified in Example 7.

### Kits

Any of the compositions described herein may be comprised in a kit. The kits will thus comprise, in suitable container means, compositions related to the present invention. In specific aspects, the kit will comprise one or more agents that target 53BP1; somatic cells; expression vectors comprising one or more agents that target 53BP1; induced pluripotent cells generated by methods disclosed herein; tissues generated by induced pluripotent cells generated by methods disclosed herein; and so forth.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present invention also will typically include a means for containing the composition and any other reagent containers in close confinement for commercial sale. Such containers may include injection or blow molded plastic containers into which the desired vials are retained.

When the components of the kit are provided in one and/or more liquid solutions, the liquid solution is an aqueous solution, with a sterile aqueous solution being particularly preferred. The compositions may also be formulated into a syringeable composition. In which case, the container means may itself be a syringe, pipette, and/or other such like apparatus, from which the formulation may be applied to an infected area of the body, injected into an animal, and/or even applied to and/or mixed with the other components of the kit. However, the components of the kit may be provided as dried powder(s). When reagents and/or components are provided as a dry powder, the powder can be reconstituted by the addition of a suitable solvent. It is envisioned that the solvent may also be provided in another container means.

### EXAMPLES

The present invention may be better understood by reference to the following non-limiting examples, which are presented in order to more fully illustrate the preferred embodiments of the invention. They should in no way be construed to limit the broad scope of the invention.

### Example 1—Materials and Methods for Examples 2-6

### Animal Breeding

To generate Bard1 mutants, heterozygous Bard1^{K607A/+} and Bard1^{S563F/+} females on a C57BL/6J background (Billing et al., 2018) were bred to males of the same genotypes. The Brca1Smarcal1 genotype collection was created from intercrosses between Brca1^{tr/+} Smarcal1^{+/-} animals of mixed C57BL/6J and 129Sv background. The Brca1tr/+ allele is described in (Ludwig et al., 2001). Mice, mutant for Smarcal1 were obtained from the International Mouse Phenotyping Consortium (IMPC). The Brca1 53bp1 combination genotype panel was generated from intercrosses between Brca1^{tr/+} 53bp1^{+/-} males and females on a mixed C57BL/6J and 129Sv background. For the Abraxas Bach1 CtIP genotype collection, referred to as ABC, mixed background (C57BL/6J and 129Sv) AABB mice to CC to generate F1 triple heterozygous A+B+C+ animals were first crossed. The F1 generation of A+B+C+ was intercrossed to obtain the different combinations of double homozygous mutants. To generate triple homozygous phosphoserine mutants, F2 A+BBCC males were crossed to F2 A+BBCC or A+BBC+ females. From the A+BBCC x A+BBCC crosses, 1 of 9 embryos was triple homozygous mutant (expected Mendelian ratio 1/4). One additional triple homozygous mutant embryo was found from the triple heterozygous intercrosses (A+B+C+x A+B+C+) which produced 69 embryos (expected Mendelian ratio 1/64).

### Fibroblast Derivation and Genotyping

To derive fibroblasts for reprogramming, E13.5 mouse embryos were harvested from the above described crosses and processed them as in (Durkin, 2013) with minor modifications. The cells from a single embryo were then plated in one 10 cm dish and grown in MEF media, consisting of DMEM HG (Thermo Fisher Scientific #10569010), supplemented with 10% FBS (Atlanta Biologicals #S11150), Glutamax (Thermo Fisher Scientific #35050079) and PenStrep (Thermo Fisher Scientific 15140163). Cells were split once to P1 and frozen down for reprogramming experiments. The sequences of all genotyping primers are provided in Table 1.

TABLE 1
Genotyping Primers

| Genotype | Forward Primer | Reverse Primer |
|---|---|---|
| Bard1^{S563F/+} | GCAGGTGCTCTACCCTCAA | AACCTGGCCATCAACATG (SEQ ID |
| Bard1^{S563F/S563F} | C (SEQ ID NO: 1) | NO: 2) |
| Bard1^{K607A/+} | CACGTGGTTGCTGGAAATT | ATGTAAAGGAGCCAGCAGC (SEQ |
| Bard1^{K607A/K607A} | G (SEQ ID NO: 3) | ID NO: 4) |
| Brca1^{tr/+} | TGCTCACTCTGTGCCCTCA | TCCATTCTCCCCGCTTCTGT (SEQ |
| Brca1^{tr/tr} | A (SEQ ID NO: 5) | ID NO: 6) |
| Smarca^{+/-} | CCGCTCTAACCTGGGAACA | GTGACAGACAACAGCCAGCC (SEQ |
| Smarcal1^{-/-} | C (SEQ ID NO: 7) | ID NO: 8) |
| | | TCGTGGTATCGTTATGCGCC (SEQ |
| | | ID NO: 9) |
| 53 bp^{+/+} | AGGAGACTGAAGAACCAA | CTCAGTTTTCCTGGGCCTCCT (SEQ |
| | TCG (SEQ ID NO: 10) | ID NO: 11) |
| 53 bp^{+/--} | GTCAGGGTTTCACTGGCTT | CCTTCTTGACGAGTTCTT (SEQ ID |
| 53 bp1^{-/-} | G (SEQ ID NO: 12) | NO: 13) |
| Abraxas^{S404A+} | CAGCAGGCACCAAGACAA | TCTGTGTATTAATCCGAGAGGCAA |
| Abraxas^{S404A/S404A} | GG (SEQ ID NO: 14) | AGA (SEQ ID NO: 15) |
| Bach^{1S994A+} | GCCAAGTGTCCCAGCTCAA | TCAGTGTCCCAGGCAACTAAG |
| Bach^{1S994A/S994A} | A | (SEQ ID NO: 17) |
| | (SEQ ID NO: 16) | |
| CtIP^{S327+} | TAGCAAAAGTCCTCAGTGG | TGTTGCTAAAGGGAGCTGTC (SEQ |
| CtIP^{S327A/S327A} | GC (SEQ ID NO: 18) | ID NO: 19 |

### Virus Preparation and Infection

This study used a doxycycline inducible lentiviral system, consisting of Tet-O-FUW-OSKM (Addgene #20321) and FUW-M2rtTA (Addgene #20342). Lentivirus was prepared in 293T cells by transfection of plasmids with Jetprime transfection reagent (VWR #89129-922) as outlined in the manufacturer's instructions. Briefly, Tet-O-FUW vectors were transfected together with the envelope and packaging plasmids from Didier Trono pMD2VSVG (Addgene #12259) and psPax2 (Addgene #12260) into 293T cells plated on collagen-coated dishes. Fresh antibiotic free media DMEM HG (Thermo Fisher Scientific #10569010), supplemented with 15% FBS (Atlanta Biologicals #S11150) and Glutamax (Thermo Fisher Scientific #35050079) was provided 16 to 20 h post transfection. Viral supernatant was collected on each of the following two days and kept at 4° C. for up to 4 days. Prior to infection, titer from the two collection days was pooled and filtered through a 40 uM cell strainer (Fisher Scientific #08-771-1).

For infection, P1 mouse embryonic fibroblasts (MEFs) were thawed and plated at 1×10⁶ cells per 10 cm dish on the previous day. Infection proceeded in two rounds with 8 h to 9 h in between. Briefly, cells were incubated with an OSKM/rtta virus mix (1:1), supplemented with 8 ug/ml protamine sulfate (Fisher Scientific #0219472905). The infection mix was removed on the following day and cells were left to recover in fresh MEF media (DMEM HG Thermo Fisher Scientific #10569010 with 10% FBS Atlanta Biologicals #S11150, Glutamax Thermo Fisher Scientific #35050079 and PenStrep Thermo Fisher Scientific 15140163).

### Reprogramming

Two days after infection, cells were re-plated for transduction efficiency assessment on day 3, molecular analyses on day 5, colony picking on day 16 and alkaline phosphatase (AP) staining on day 20. In each experiment, infected fibroblasts from the different genotypes were replated at multiple densities to allow for optimal reprogramming efficiency. For wild-type cells, 100-300 cells/mm² (20-60K per well of a 24 w dish) routinely generated high numbers of iPS cell clones. Besides for wild-type, 20-60K per well of a 24 w dish was also optimal for the Bard1 point mutants, Brca1^{tr/+} and all combination mutations with Smarcal1 or 53bp1; Brca1^{tr/tr}, 53bp1^{-/-} as well as the heterozygous or homozygous Smarcal1 and 53bp1 single mutants. The 3 genotypes Brca1^{tr/tr}; Brca1^{tr/tr}Smarcal1^{+/-} and Brca1^{tr/tr}Smarcal1^{-/-} were plated at 600-800 cells/mm² (120-160K per well of a 24 w dish) since no iPS clones were observed at the densities selected for wild-type. The remaining Brca1^{tr/tr}53BP1^{+/-} genotype was re-plated at 450 cells/mm² (90K/well of a 24 w dish). These seeding densities were later used to calculate the reprogramming efficiency of each genotype. The OSKM reprogramming factors were induced with 1 ug/ml doxycycline (Sigma #D9891) in mouse embryonic stem (mES) cell media, consisting of Knockout DMEM (Life Technologies #10829-018), supplemented with 15% Knockout Serum Replacement (Life Technologies #10828-028), Glutamax (Thermo Fisher Scientific #35050079), MEM NEAA (Life Technologies #11140050), PenStrep (Thermo Fisher Scientific 15140163), 2-mercaptoetahnol (Life Technologies #21985-023) and 10 ng/ul LIF (eBioscience #34-8521-82). Transduction efficiency was determined on reprogramming day 3 by staining for Sox2 (Stemgent #09-0024) and used in the calculation of reprograming efficiency. The reprogramming experiments with drug treatment used aphidicolin (Sigma #A0781) at 0.2 uM, Ttpotecan (Sigma #T2705) at 10 nM or the DNA PK inhibitor NU7026 (Tocris #2828) at 5 uM for 8 days during reprogramming.

Alternatively, for the induction of two-ended DSBs, cells were subjected to a single dose of 6 Gy IR 1 day post doxycycline-mediated OSKM factor induction. Cells were fixed on reprogramming day 18-20 and stained for alkaline phosphatase with the Vector Red detection kit (Vector Laboratories #SK-5100). Reprogramming efficiency was determined by considering the number of AP-positive colonies per number of infected cells, determined by Sox2 staining at the optimal plating density for each genotype. The sensitivity score in the experiments with aphidicolin, topotecan and IR was calculated as follows: the reprogramming efficiency of wild-type treated cells was determined as described above and normalized to the reprogramming efficiency of wild type untreated cells. The same procedure was applied to each mutant genotype. The sensitivity score was obtained by calculating the ratio of treated wild type (normalized to untreated wild type) to treated mutant (normalized to untreated mutant). A large score corresponds to high sensitivity.

### Immunofluorescence, Western Blot and DNA Fiber Analysis

Detection of TH2AX, phospho RPA(S33) and 53bp1 was performed on reprogramming D5 with the following antibodies: Phospho-RPA2Ser33 (Invitrogen #PA5-39809); Anti-phosphoHistone H2A.X-Ser139 (Millipore #05-636); and 53BP1 Antibody H-300 (Santa Cruz #22760). For detection of Rad51, iPS cell lines were irradiated with 10 Gy and stained with Rad51 (Ab-1) Rabbit pAb (Millipore #PC130) 1.5 h post IR.

For Western Blot, E13.5 wild-type and 53bp1 mutant MEFs were subjected to 8 Gy of IR and harvested 6 h post treatment. Lysis was performed in RIPA buffer and proteins of interest were detected with the following antibodies-p21 (Abcam #ab188224) and α-tubulin (Abcam #ab4074).

DNA fiber analysis on Brca1, Smarcal1 and 53bp1 combination mutants during reprogramming was carried out as described in Terret et al., 2009. Briefly, fibroblasts of different genotypes were incubated on reprogramming day 5 with 25 uM CldU for 30 min, washed 3 times with warm PBS and incubated with 125 uM IdU for another 30 min. Fork stalling was then induced by a 5 h-long treatment with 2 mM hydroxyurea (HU). For the ABC genotype collection and for the Brca1tr/+ genotype, immortalized uninfected fibroblasts were incubated with IdU for 20 min, followed by a wash and CldU for 20 min. Fork stalling was induced with 2 mM HU for 1.5 h. Fibers were stretched on slides and stained with anti-BrdU/CldU (Biorad #OBT0030) and anti-BrdU/IdU (BD #347580) antibodies. Imaging was performed with a 100× objective on an Olympus microscope and fiber length was measured with Olympus cellSens imaging and analysis software. In an alternative fiber assay, fork stalling was induced by treatment with 2 uM pyridostatin (PDS) during the 30 min incubation with 125 uM IdU.

### HR Assay

The HR competence of the different genotypes was evaluated in mouse induced pluripotent stem (iPS) cells with a CRISPR/Cas9-based assay where a zsGReen repair template is targeted to the Hsp90 genomic locus. This strategy has been described in detail by Mateos-Gomez et al., 2017. In short, 200-300×103 exponentially growing iPS cells were transfected with 200 ng Cas9-puromycin vector and 800 ng zsGreen repair template with Jetprime transfection reagent (VWR #89129-922) as outlined in the manufacturer's instructions. Media was changed approximately 20 h post transfection for 24 h. To enrich for Cas9-transfected cells, the plates were treated with 1 ug/ml puromycin (Thermo Fisher #A11138-03) for approximately 20 h. Flow cytometry for zsGreen was performed on the 3rd day of recovery form puromycin selection. To exclude potentially non-transfected cells, the efficiency of single versus dual allele targeting was compared.

### Proliferation and Apoptosis

To evaluate proliferation, infected fibroblasts on reprogramming D2 were incubated with 5 uM Cell Trace CSFE proliferation dye (Thermo Fisher #C34554) for 20 min at 37° C. as outlined in the manufacturer's protocol. Cells were then changed to fresh mouse ES cell media, composed of Knockout DMEM (Life Technologies #10829-018), 15% Knockout Serum Replacement (Life Technologies #10828-028), Glutamax (Thermo Fisher Scientific #35050079), MEM NEAA (Life Technologies #11140050), PenStrep (Thermo Fisher Scientific 15140163), 2-mercaptoetahnol (Life Technologies #21985-023) and 10 ng/ul LIF (eBioscience #34-8521-82), supplemented with 1 ug/ml doxycycline (Sigma #D9891). Three days post incubation with CSFE (reprogramming day 5) cells were harvested for flow cytometry.

For apoptosis analysis, cells were collected on reprogramming day 5 and stained without fixation with the Annexin V-FITC apoptosis detection kit (Sigma #APOAF-20TST) according to protocols provided by the manufacturer. The numbers of early and late apoptotic cells were determined by flow cytometry for Annexin V-FITC and propidium iodide (PI). Early apoptosis is marked by Annexin V staining only, while late apoptotic cells stain for both Annexin V and PI.

### Example 2—Reprogramming Depends on the Interaction of BRCA1 with its BRCT Domain Phospholigands, Abraxas, Bach1, and CtIP

It was previously reported that reprogramming is severely impaired in mouse fibroblasts that are homozygous for either of two pathogenic Brca1 lesions: Brca1^{tr/tr} and Brca1^{S1598F/S1598F}(Gonzalez et al., 2013). While the Brca1tr allele encodes a C-terminally-truncated protein that lacks several critical BRCA1 domains, including its SQ cluster region, PALB2-binding sequence, and BRCT motif (Ludwig et al., 2001), the protein product of Brca1S1598F harbors a missense mutation that specifically disrupts the phosphate-binding cleft of the BRCT domain (Shakya et al., 2011). By virtue of its BRCT phosphor-recognition domain, BRCA1 can interact, in a mutually exclusive manner with the phosphorylated isoforms of several DNA repair factors, including ABRAXAS, BACH1/BRIP1/FANCJ, and CtIP (Cantor et al., 2001; Wang et al., 2007; Yu, 1998). Since its interaction with each of these BRCT phosphor ligands is mutually exclusive, BRCA1 can form multiple distinct in vivo protein complexes that appear to mediate various aspects of BRCA1 function (e.g., BRCA1 complexes A, B, and C).

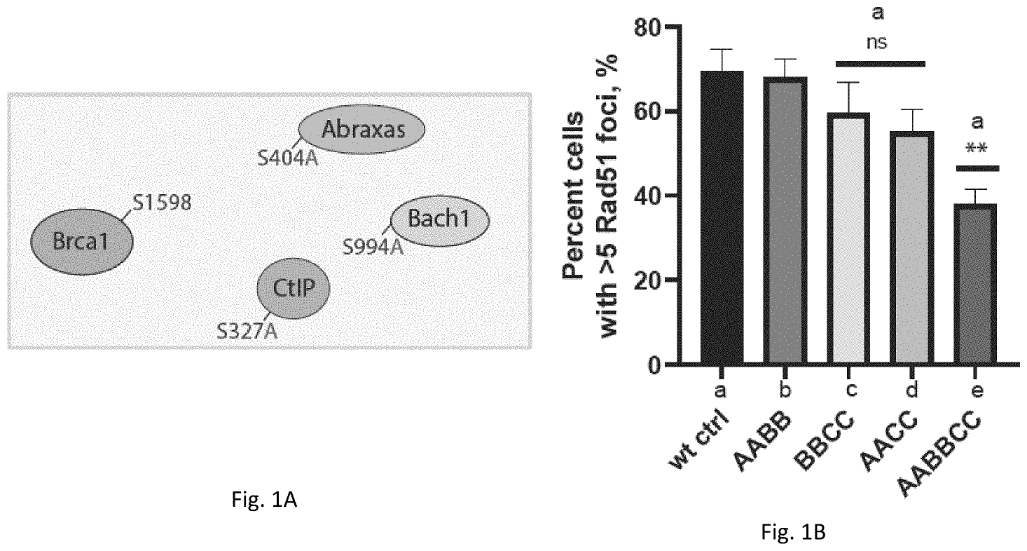

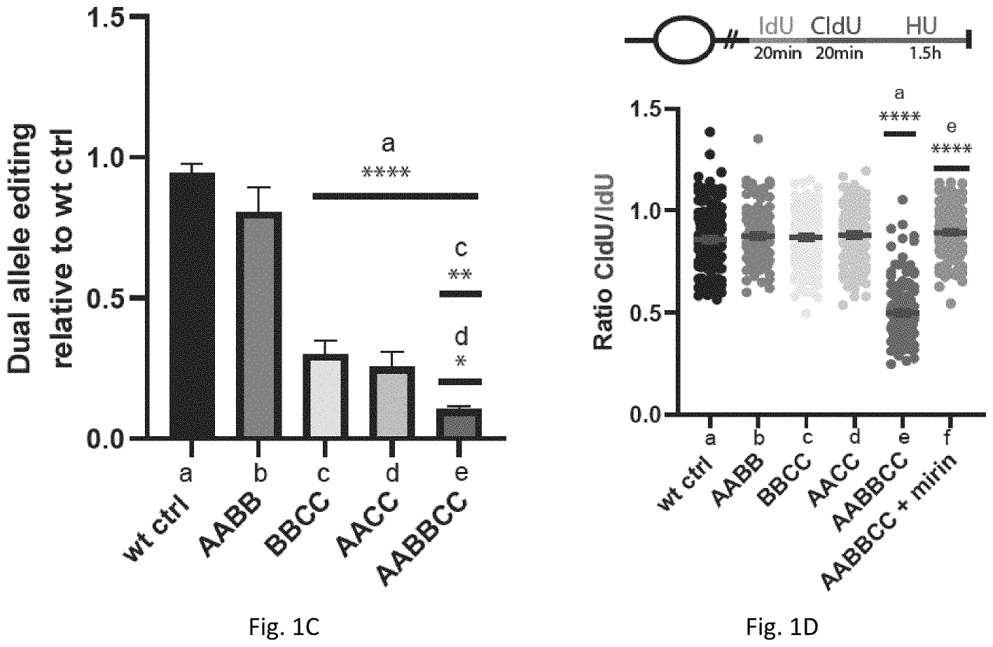

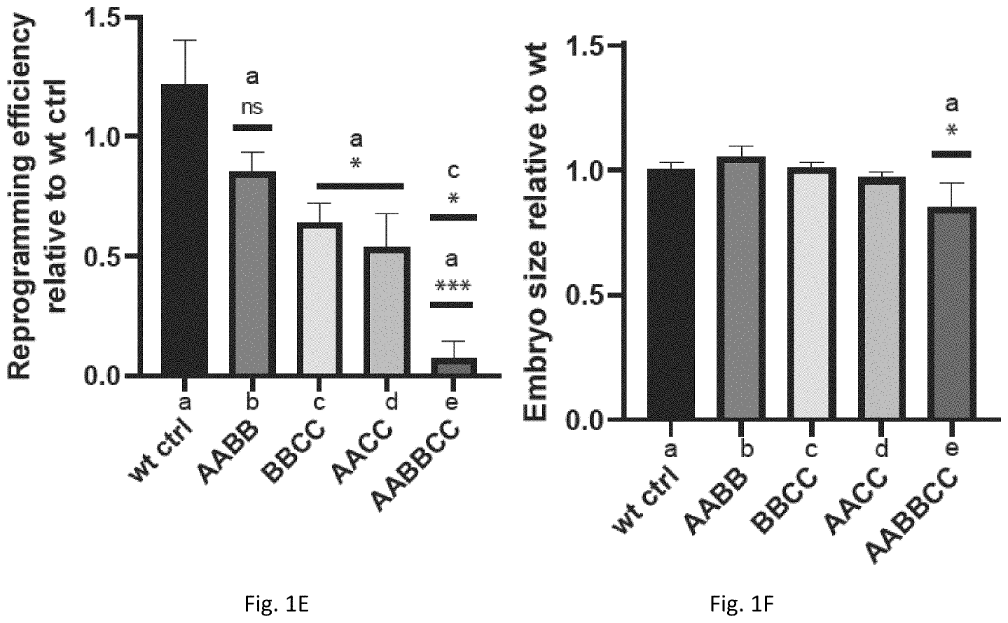

To test whether the interaction of BRCA1 with one or more of its BRCT phospho-ligands is required for reprogramming, mouse embryonic fibroblasts (MEFs) that are homozygous for serine-to-alanine substitutions in the critical phosphorylation sites of Abraxas (S404A), Bach1 (S994A) and/or Ctip (S327) were examined (FIG. 1A). Previous studies of Brca1^{S1598F/S1598F} cells revealed that BRCT phospho-recognition is required for both HDR (Shakya et al., 2011) and SFP (Billing et al., 2018) as well as for reprogramming (Gonzalez et al., 2013). To ascertain whether these functions of Brca1 are dependent on its interactions with Abraxas, Bach1, and/or Ctip, MEFs and iPS cell lines harboring combinations of homozygous AA, BB, and CC missense mutations were examined. To measure HDR function, ionizing radiation-induced focus (IRIF) formation of the Rad51 recombinase was examined by immunofluorescent microscopy in wild type and mutant cell lines 1.5-hour post 10 Gy irradiation. As shown in FIG. 1B, while Rad51 IRIFs were generated at comparable levels in double-mutant MEFs (AABB, BBCC, and AACC), IRIF formation was severely impaired in triple-mutant AABBCC. The genotypes with Ctip point mutations BBCC and AA CC had lower numbers of Rad51 foci, but the difference was not significant (FIG. 1B).

To examine the HDR competence of BBCC and AACC mutants further, a CRISPR/Cas9-based assay which targets the Hsp90 locus in the mouse genome and provides a zsGreen-containing repair template for HDR was utilized (Mateos-Gomez et al., 2017). This experiment revealed a marked reduction in the capacity of BBCC and AACC for HDR, which was most severe in the triple homozygous mutant AABBCC (FIG. 1C). The difference was most apparent when considering dual allele edited cells, which form a distinct, fluorescently brighter population (FIG. 1C). To measure SFP function, MEFs were treated with hydroxyurea (HU) to induce stalling of DNA replication, and the stability of stalled forks was assessed by DNA fiber analysis. As expected, and similarly to Brca1^{S1598F/S1598F} mutants (Billing et al., 2018), HU-treated AABBCC cells failed to protect HU-stalled replication forks, as indicated by the marked reduction in the ratios of CldU/IdU track lengths relative to wildtype cells (FIG. 1D).

To evaluate reprogramming efficiencies, primary wild type and mutant MEFs were infected with doxycycline inducible lentiviruses encoding the OSKM factors and stained for alkaline phosphatase (AP), an early marker of pluripotency 20 days post factor induction. All Ctip phosphoserine point mutant genotypes-BBCC, AACC and AABBCC had lower numbers of AP-positive colonies, compared to the control and this difference was most apparent in the case of the triple homozygous mutant (FIG. 1E). The reprogramming efficiency of AABBCC dropped approximately 17-fold, phenocopying the result which was previously reported for Brca1^{tr/tr} and Brca1^{S1598F/S1598F} (Gonzalez et al., 2013). Besides in reprogramming, triple homozygous point mutants were also challenged in development as indicated by the reduced size of E13.5 AABBCC embryos (FIG. 1F).

Taken together, these data indicated that disruption of BRCA1 complex C in combination with either A or B reduces the efficiency of HDR and impairs reprogramming. The simultaneous inactivation of all three BRCA1 complexes A, B and C leads to disruption of both HDR and SPF, which further exacerbates the poor reprogramming phenotype.

### Example 3—Loss of Stalled Fork Protection (SFP) does not Affect Reprogramming Efficiency

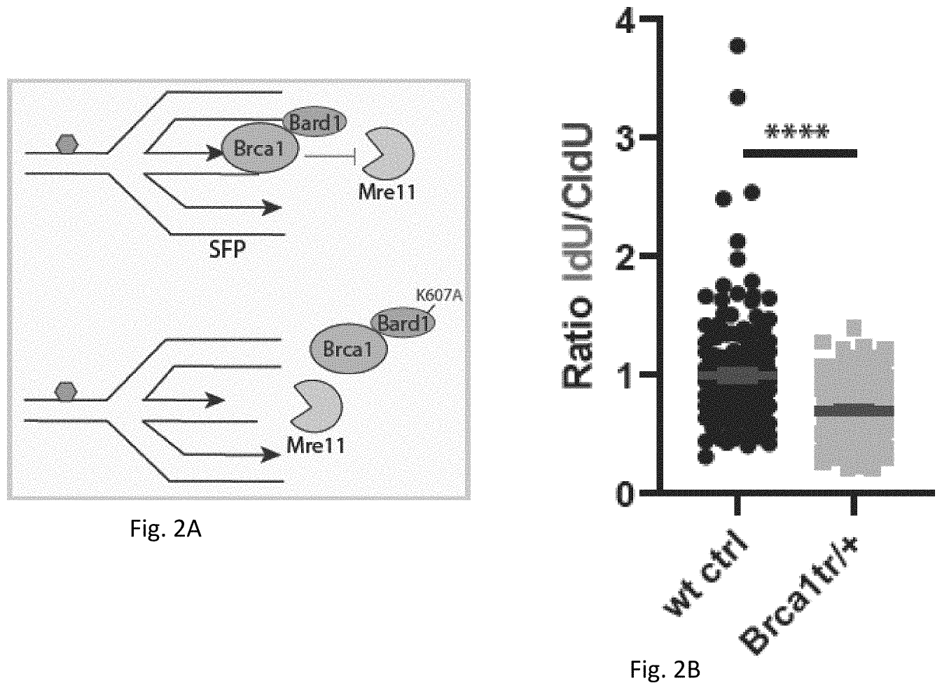

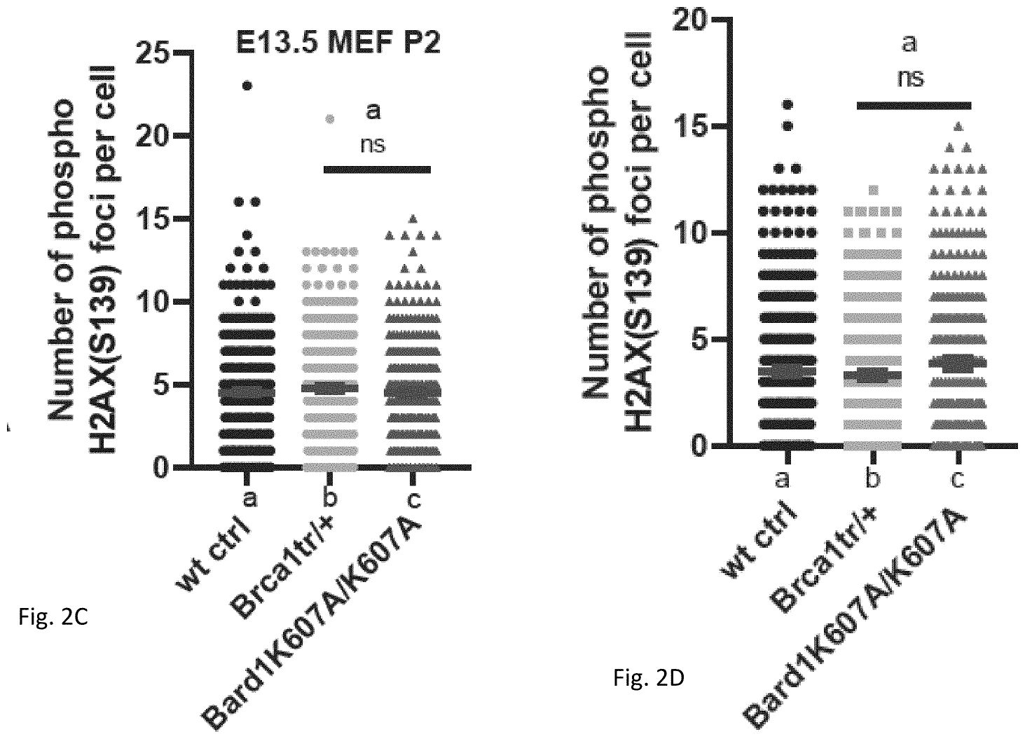

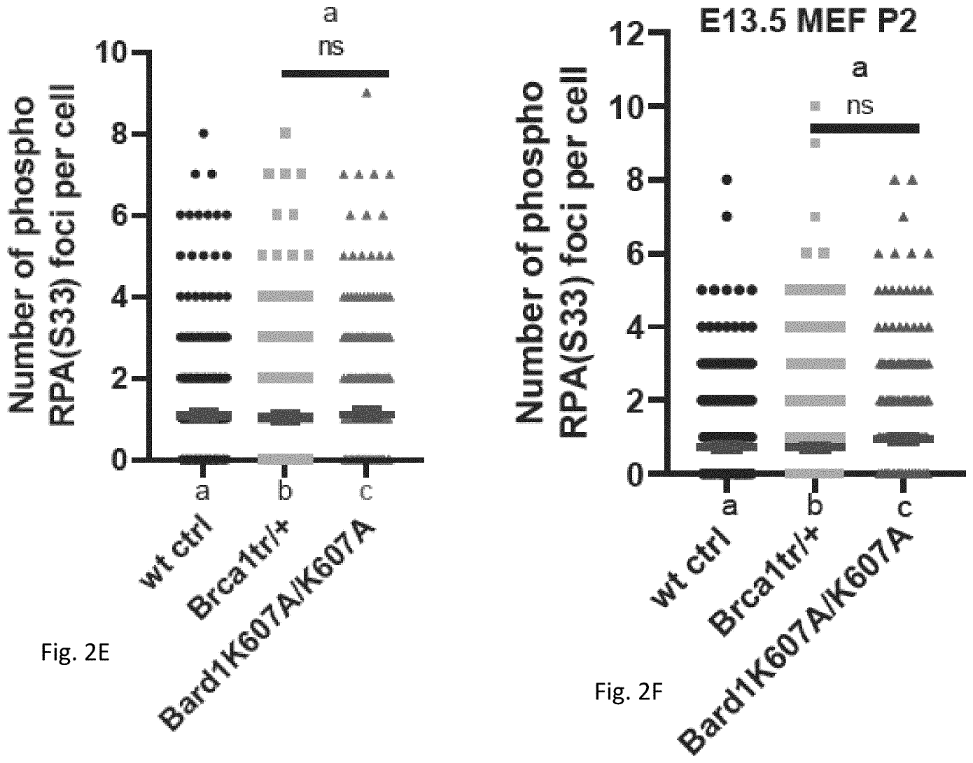

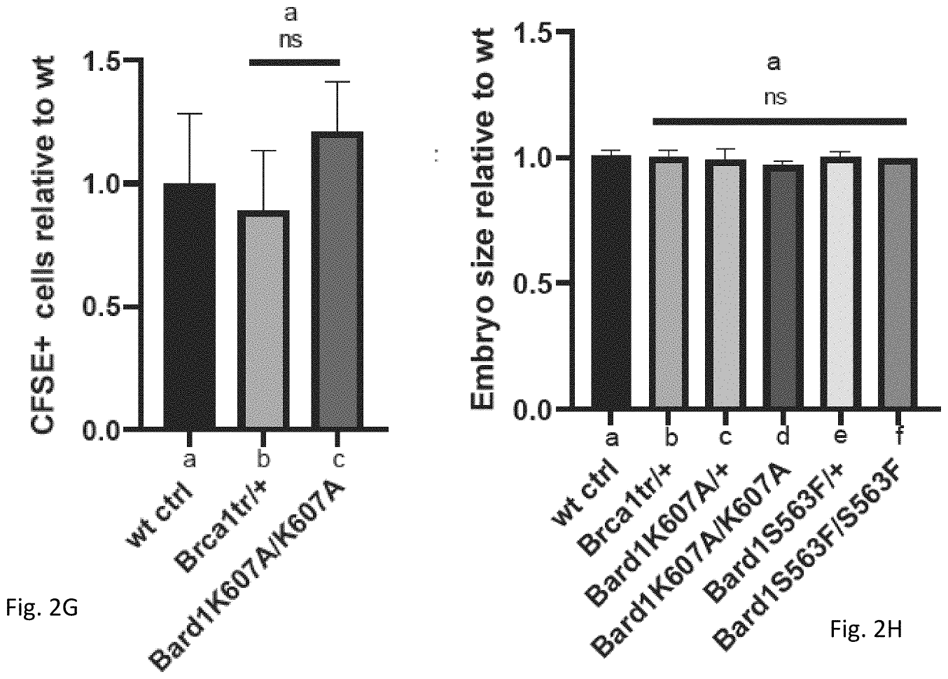

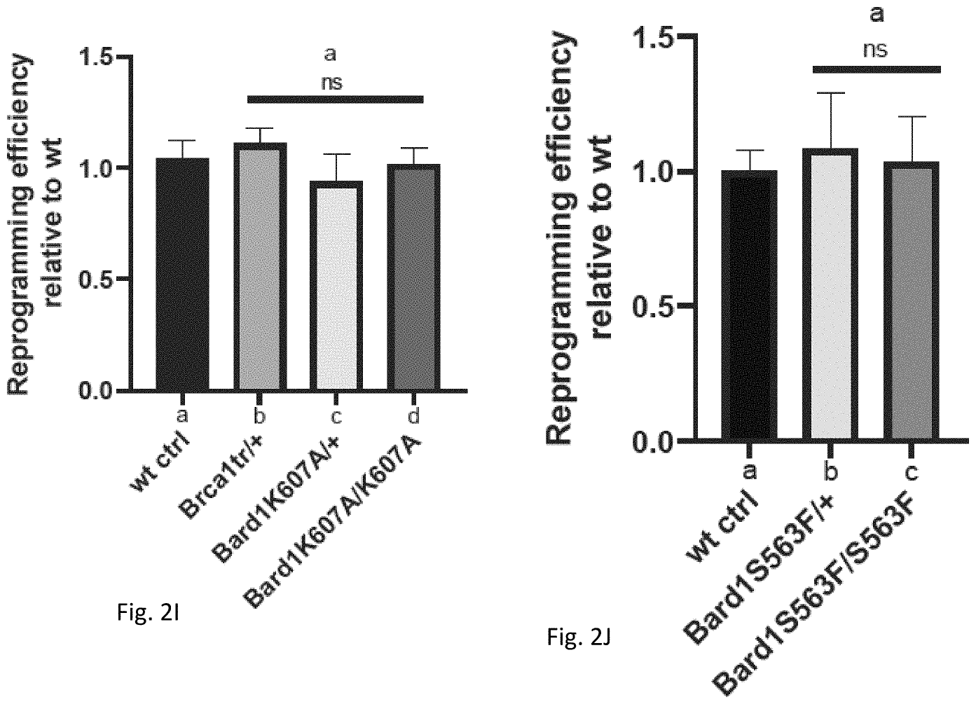

Since DNA damage can be generated by defects in either HDR or SFP, it was sought to determine which of these processes is required for efficient somatic cell reprogramming. In vivo, BRCA1 exists as a heterodimer with BARD1, a related protein that harbors a C-terminal BRCT domain with distinct phosphate recognition properties (Wu et al., 1996). To ascertain whether SFP is required for reprogramming, cells that display the HDR+SFP− phenotype were examined because 1) they carry a homozygous Bard1 separation-of-function mutation or 2) they are heterozygous for a Brca1 or Bard1 mutation. First, since Bard1K607A and Bard1S563F are separation-of-function mutations that specifically abrogate SFP without affecting HDR, Bard1^{K607A/K607A} and Brca1^{S1598F/S1598F} cells exhibit the HDR+SFP− phenotype (Billing et al., 2018) (FIG. 2A and Table 2). Second, although most biological functions attributed to BRCA1, including HDR, are unaffected in cells that are heterozygous for tumor-associated BRCA1 mutations, recent work has shown that SFP is impaired in cells that are heterozygous for certain BRCA1/BARD1 lesions. For example, Brca1^{tr/+} cells are unable to protect hydroxyurea-induced stalled replication forks from degradation as shown on FIG. 2B. In addition, Bard1^{K607A/+} and Bard1^{S563F/+} MEFs also display this HDR+SFP− phenotype (Billing et al., 2018).

To assess the consequences of SFP deficiency on DNA damage during reprogramming, the appearance of nuclear foci for phospho-H2AX(S139), known as TH2AX and phospho-RPA(S33) was quantified on day 5 of reprogramming. It was previously shown that OSKM-mediated reprogramming in wild-type cells induces a significant increase in the numbers of TH2AX foci, which serves as an indirect measure of DNA double-strand break (DSB) formation (Gonzalez et al., 2013). Similar numbers of TH2AX foci were observed in Brca1^{tr/+} and Bard1^{K607A/K607A} MEFs without infection (FIG. 2C) and during reprogramming (FIG. 2D), indicating that loss of SFP does not affect the levels of DNA damage that accrue during reprogramming. The RPA/ssDNA filaments that form as a consequence of replication stress are phosphorylated by the ATR kinase on serine 33 of the RPA2 polypeptide (Murphy et al., 2014). As shown in FIG. 2E, Brca1^{tr/+} and Bard1^{K607A/K607A} cells did not display an increase in the numbers of phospho(S33)-RPA2 foci relative to wild-type cells, indicating that SFP loss does not exacerbate replication stress during reprogramming (FIG. 2E and FIG. 2F). The proliferation rates of HDR+SFP− cells on day 5 of reprogramming were indistinguishable from those of wild-type cells, as measured by CFSE retention (FIG. 2G), while the size and morphology of HDR+SFP− embryos at day E13.5 were also normal (FIG. 2H). Most importantly, the reprogramming efficiencies of all HDR+SFP− cells (Brca1tr/+, Bard1^{K607A/K607A} Bard1^{K607A/+}, Bard1^{S563F/+} and Bard1^{S563/S563F}), measured by the number of alkaline phosphatase (AP) positive colonies on day 20 post factor induction, were indistinguishable from those of wild-type cells, regardless of genotype (FIG. 2I and FIG. 2J). Thus, the loss of SFP does not impair the efficiency of reprogramming.

TABLE 2
Reprogramming Genotype Collection

| | | γH2AX | RPA(S 33) | 53bp | | | Cancer | |
|---|---|---|---|---|---|---|---|---|
| Genotype | Phenotype | foci | foci | 1 foci | Apoptosis | Reprog. Eff. | Suscep. | Embryo size |
| Bard1^{K607A/+} | HDR+ | | | | | Same | No | Same |
| | SFP− | | | | | as wt | | as wt |
| Bard1^{K607A/K607A} | HDR+ | Same | Same | Same | | Same | No | Same |
| | SFP− | as wt | as wt | as wt | | as wt | | as wt |
| Bard1^{S563F/+} | HDR+ | | | | | Same | No | Same |
| | SFP− | | | | | as wt | | as wt |
| Bard1^{S563F/563F} | HDR+ | | | Same | | Same | No | Same |
| | SFP− | | | as wt | | as wt | | as wt |
| Brca1^{tr/+} | HDR+ | Same | Same | Same | | Same | No | Same |
| | SFP− | as wt | as wt | as wt | | as wt | | as wt |
| Smarcal1^{+/-} | HDR+ | | | | Same | Same | | Same |
| | SFP+ | | | | as wt | as wt | | as wt |
| Smarcal1^{-/-} | HDR+ | | | | Same | Same | | Same |
| | SFP+ | | | | as wt | as wt | | as wt |
| Brca1^{tr/+}Smarcal1^{+/-} | HDR+ | | | | | Same | | Same |
| | SFP−* | | | | | as wt | | as wt |
| Brca1^{tr/+}Smarcal1^{-/-} | HDR− | | | | | Same | | Same |
| | SFP+ | | | | | as wt | | as wt |
| Brca1^{tr/tr} | HDR- | ↑↑↑ | ↑↑↑ | ↑↑↑ | ↑↑↑ | ↓↓↓ | Yes | ↓ |
| | SFP- | | | | | ↓↓↓ | | |
| Brca1^{tr/tr}Smarcal1^{+/-} | HDR- | | | | | ↓↓↓ | | ↓ |
| | SFP-* | | | | | ↓↓↓ | | |
| Brca1^{tr/tr}Smarcal1^{-/-} | HDR- | Same | ↑ | ↑↑↑ | ↑↑↑ | ↓↓↓ | | ↓ |
| | SFP+ | as wt | | | | | | |
| 53bp^{+/-} | HDR+ | | | | Same | Same | | Same |
| | SFP+ | | | | as wt | as wt | | as wt |
| 53bp1^{-/-} | HDR+ | | | | Same | ↑ | Yes | Same |
| | +SFP+ | | | | as wt | | | as wt |
| Brca1^{tr/+}53bp1^{-/-} | HDR+ | | | | | ↑ | | Same |
| | +SFP−* | | | | | | | as wt |
| Brca1^{tr/tr}53bp1^{-/-} | HDR+ | Same | Same | None | Same | Same | No | Same |
| | SFP+# | as wt | as wt | | as wt | as wt | | as wt |
| AABB | HDR+ | | | | | Same | No | Same |
| | SFP+ | | | | | as wt | | as wt |
| BBCC | HDR− | | | | | ↓↓ | No | Same |
| | SFP+ | | | | | | | as wt |
| AACC | HDR− | | | | | ↓↓ | No | Same |
| | SFP+ | | | | | | | as wt |
| AABBCC | HDR− | | | | | ↓↓↓ | No | ↓ |
| | SFP− | | | | | | | |

*To be confirmed
#improved but not completely rescued

### Example 4—Restoring Fork Protection Brca1 Mutant Cells Reduces DNA Damage, but does not Improve Reprogramming Efficiency

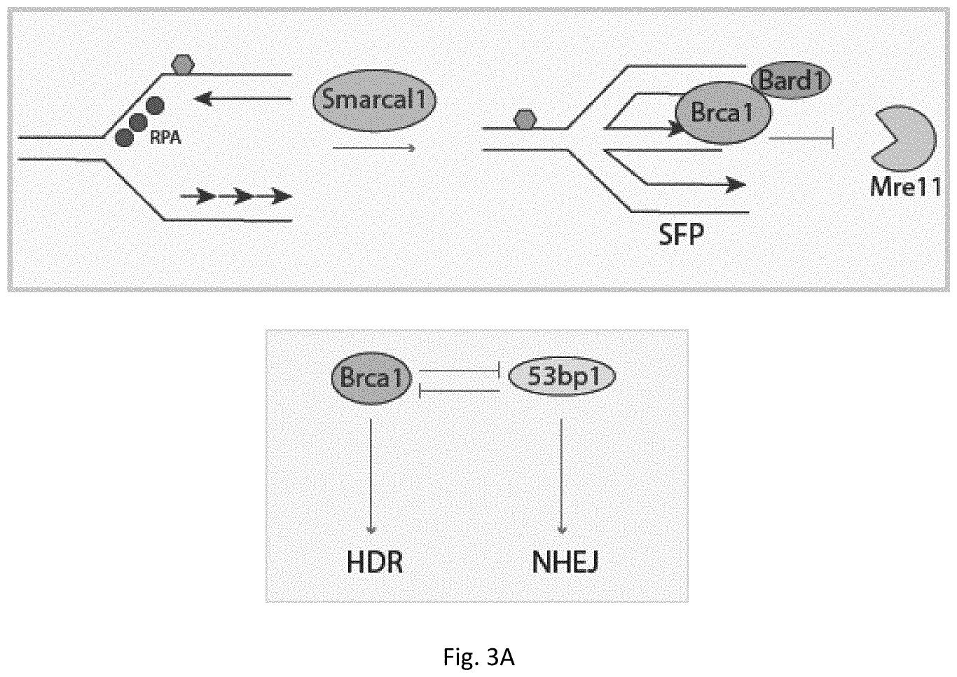

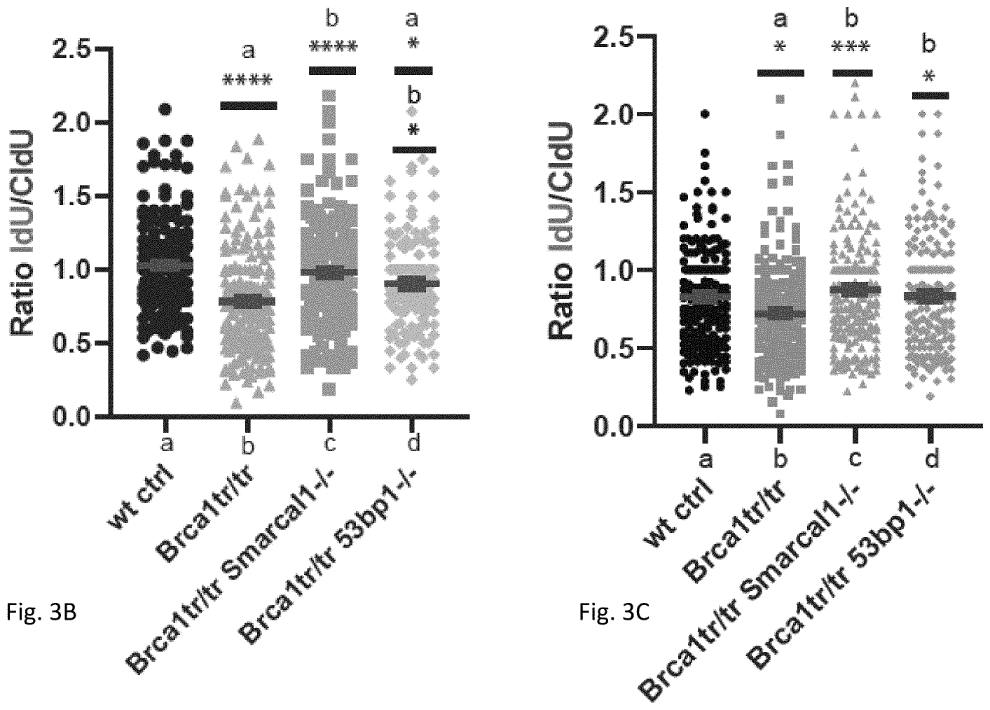

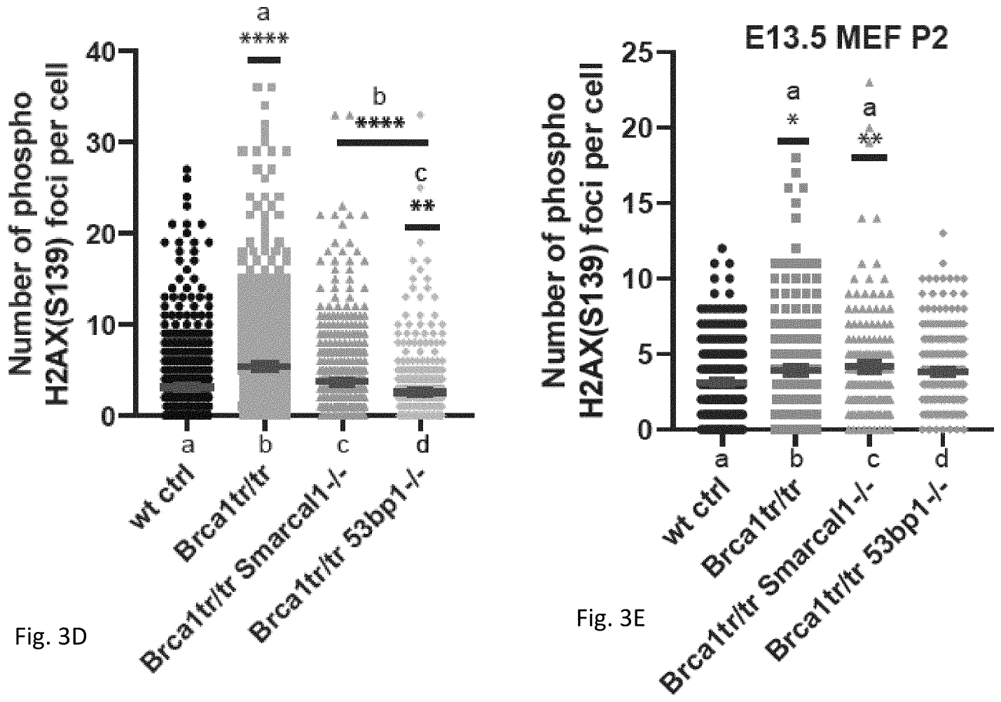

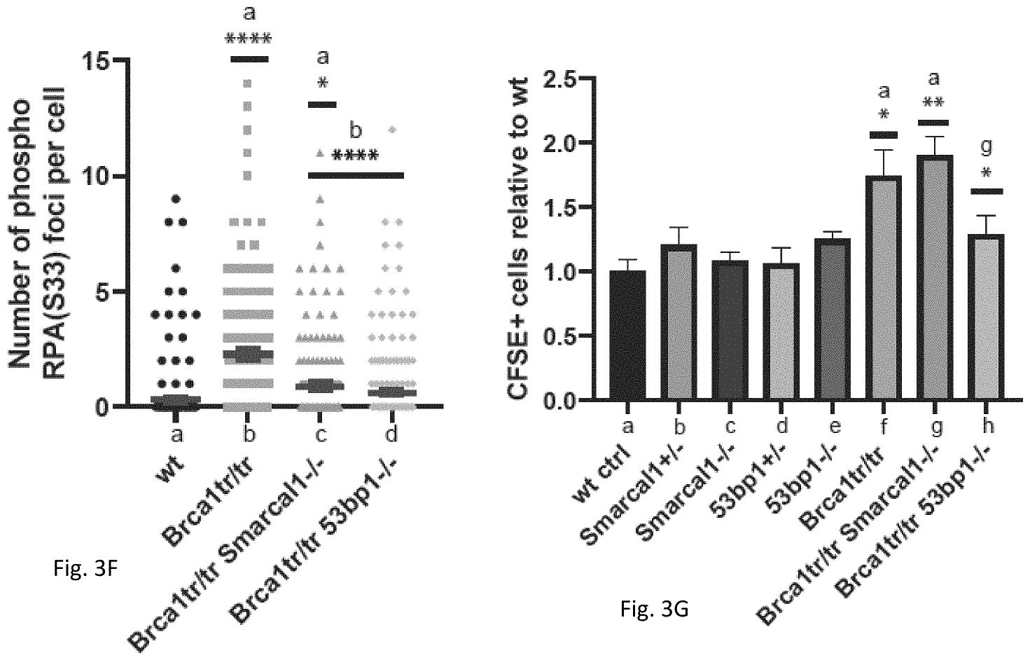

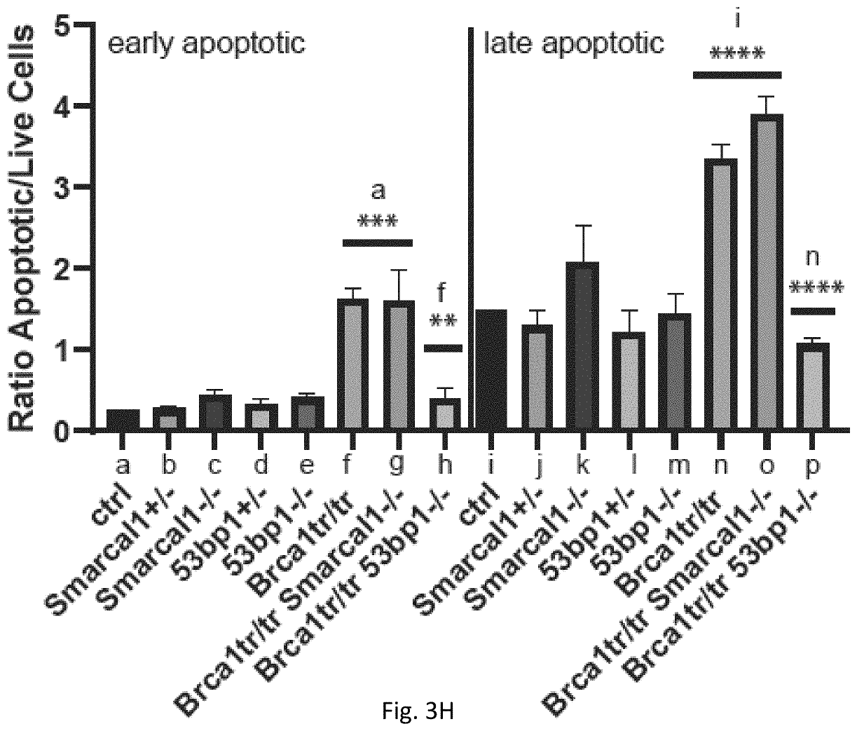

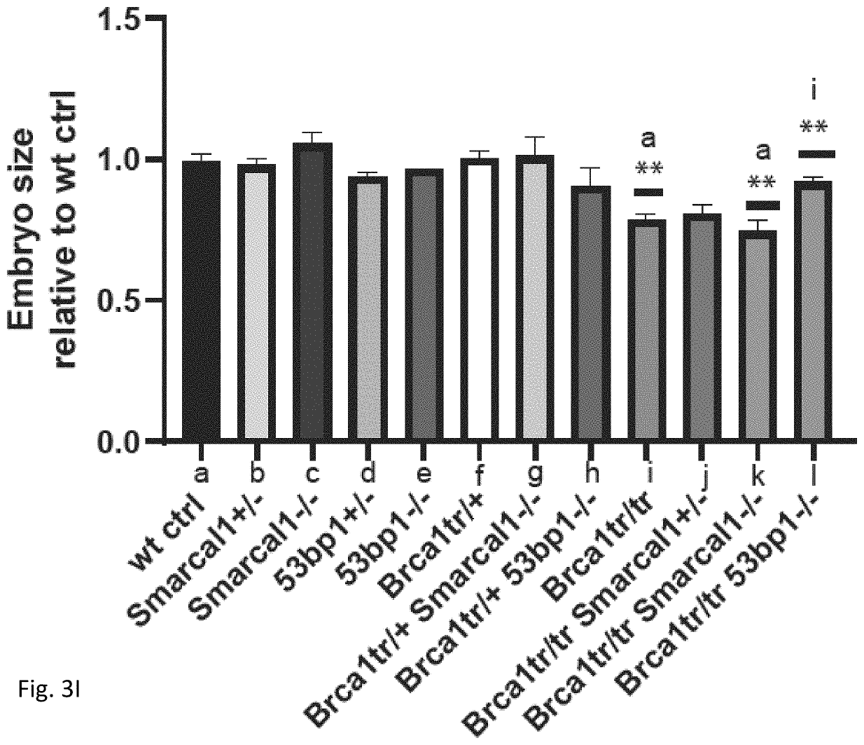

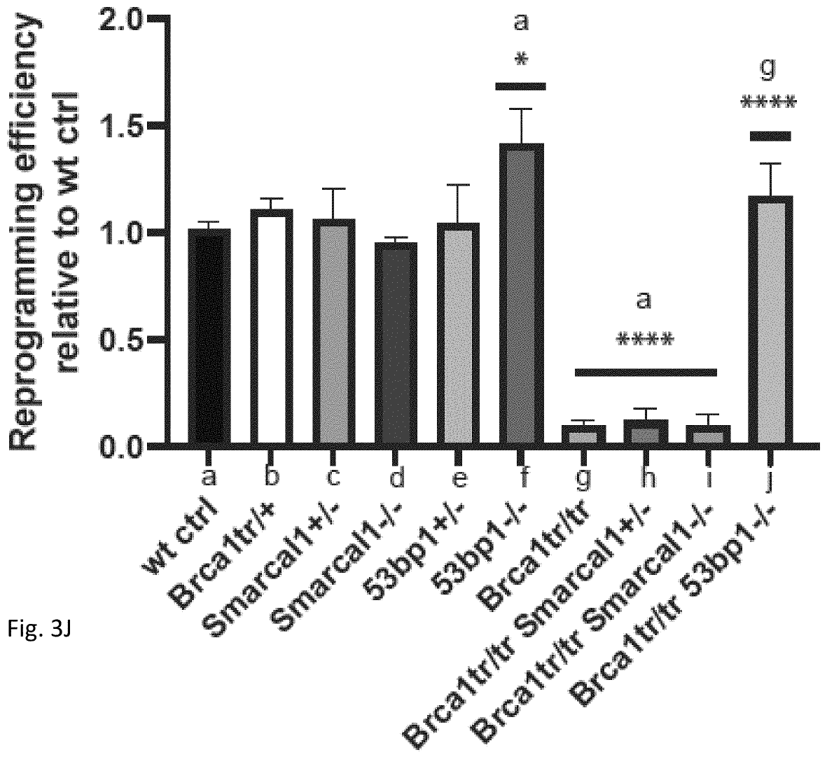

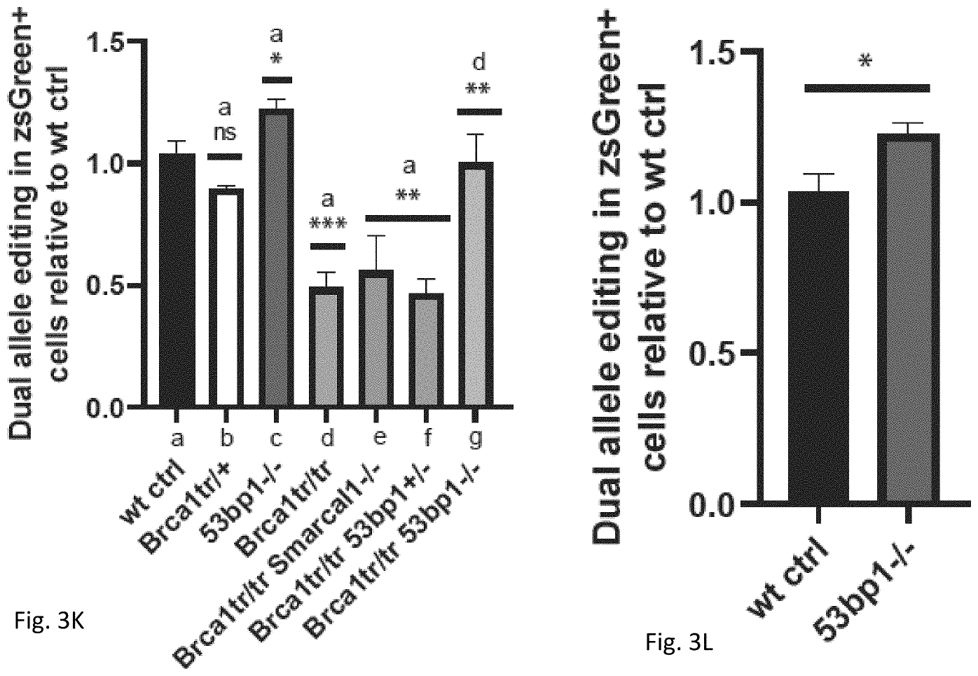

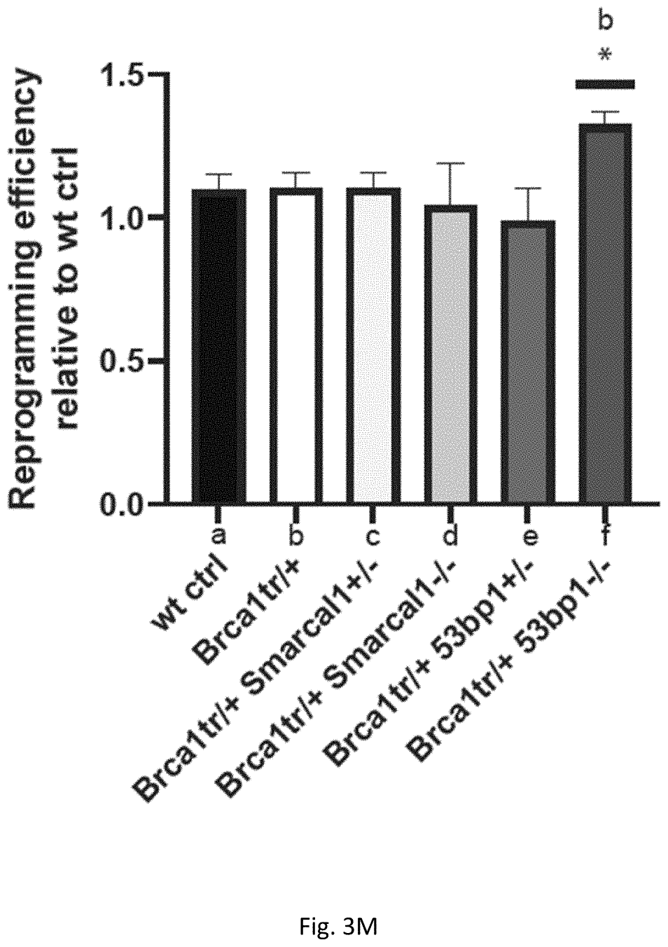

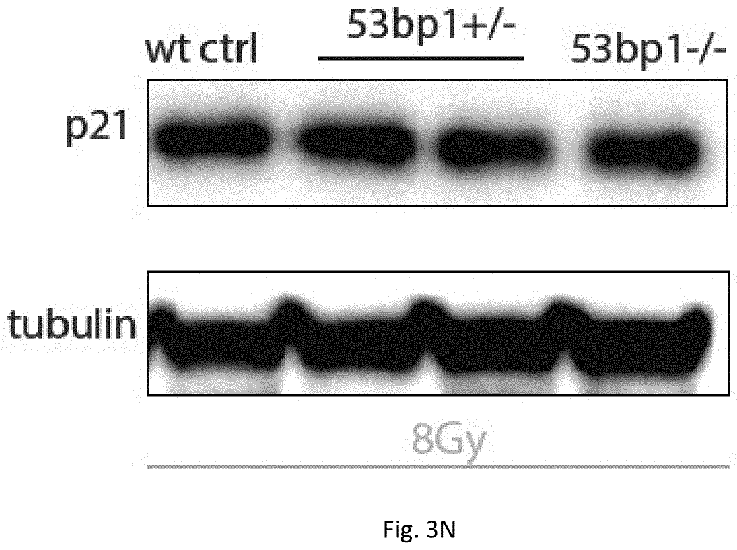

To ascertain whether reprogramming is dependent on the HDR function of Brca1, Brca1 mutant cells that exhibit the HDR-SFP+ phenotype due to loss of the Smarcal1 DNA translocase were examined. The SMARCAL1-related family of DNA translocases (SMARCAL1, ZRANB3, and HTLF) are required to remodel newly stalled replication forks into the reversed (regressed or ‘chicken-foot’) fork, an intermediate structure that normally facilitates fork restart by template switching (FIG. 3A). Since reversed forks serve as the substrates for Mre11-dependent fork degradation in Brca1-mutant cells, Smarcal1 inhibition can specifically rescue SFP, but not HDR, in these cells (Taglialatela et al., 2017). Thus, while Brca1^{tr/tr} cells display the HDR-SFP− phenotype, Brca1^{tr/tr}Smarcal1^{-/-} cells should be proficient for SFP.

To confirm that SFP is restored in Brca1^{tr/tr}Smarcal1^{-/-} cells during somatic cell reprogramming, DNA fiber analysis was performed using cells that had been sequentially pulse labeled with CldU and IdU prior to treatment with hydroxyurea (HU) to induce fork stalling. As shown in FIG. 3B, the mean ratio of IdU/CldU tract lengths is markedly reduced in Brca1^{tr/tr} cells relative to wild-type, indicating excessive degradation of stalled forks. However, in reprogramming Brca1^{tr/tr}Smarcal1^{-/-} cells, the IdU/CldU ratios were restored to the levels observed in wild-type cells. The SFP proficiency of Brca1^{tr/tr}Smarcal1^{-/-} cells was further established by DNA fiber analysis using, in lieu of HU, the G-quadruplex stabilizing compound pyridostatin (PDS), which stalls replication forks in G-rich regions of the genome (FIG. 3C). On day 5 of reprogramming, the numbers of TH2AX foci were markedly higher in Brca1^{tr/tr} cells than in wild-type cells (FIG. 3D), a difference which was mild in uninfected MEFs (FIG. 3E), but exacerbated after OSKM factor induction. Interestingly, TH2AX focus formation in Brca1^{tr/tr}Smarcal1^{-/-} cells was significantly lower than in Brca1^{tr/tr} B (FIG. 3D), indicating that restoration of SFP improves genomic stability in Brca1 mutants. Similarly, Brca1^{tr/tr}Smarcal1^{-/-} cells displayed lower levels of replication stress, as measured by the assembly of nuclear phospho(S33)-RPA2 foci, compared to Brca1^{tr/tr} (FIG. 3F). On day 5 of reprogramming, Smarcal1^{-/-} cells resembled the wild-type, but Brca1^{tr/tr} fibroblasts displayed proliferation impairment (FIG. 3G) and increased levels of apoptosis (FIG. 3H). Although Brca1^{tr/tr} Smarcal1^{-/-} MEFs are proficient for SFP (FIG. 3B), they also exhibited growth and viability defects comparable to those of Brca1^{tr/tr} cells (FIG. 3G, FIG. 3H). Moreover, Brca1^{tr/tr} and Brca1^{tr/tr}Smarcal1^{-/-} embryos were both significantly smaller on day E13.5 than either wild-type or Smarcal1 embryos (FIG. 3I). As shown in FIG. 3J, Smarcal1^{-/-} cells readily undergo OSKM-mediated reprogramming with an efficiency similar to wild-type MEFs. Importantly, however, Brca1^{tr/tr} Smarcal1^{-/-} cells, which have the HDR-SFP+ phenotype, displayed a severe defect in iPS cell generation (greater than 11-fold reduction), which resembled that of HDR-SFP− cells, such as Brca1^{tr/tr} and AABBCC (FIG. 1E). These results indicated that loss of HDR is sufficient to abrogate reprogramming, even in Brca1 mutant cells that are proficient for SFP.

### Example 5—Ablation of 53bp1 Rescues Brca1 Deficiency and Restores Reprogramming Efficiency by Increasing HDR

To rescue HDR in Brca1 mutants, ablation of 53bp1 was used, which has been previously reported to re-establish HDR proficiency (Bunting et al., 2010) (FIG. 3A). The rescue of HDR competence in Brca1^{tr/tr}53bp1^{-/-} cells was confirmed with a CRISPR/Cas9-based HDR assay (Mateos-Gomez et al., 2017), showing a 2-fold reduction in dual allele targeting in the Brca1^{tr/tr} genotype, which remained unchanged upon loss of Smarcal1, but was restored in the absence of 53bp1 (FIG. 3K). Notably, ablation of 53bp1 in wild type cells also resulted in enhancement of HDR capacity (FIG. 3K, FIG. 3L). Interestingly, ablating 53bp1 in Brca1 mutant cells also partially rescued their SFP defect (FIG. 3B). On reprogramming day 5, the numbers of both TH2AX and phospho RPA(S33) foci in Brca1^{tr/tr}53bp1^{-/-} B cells was significantly lower than in Brca1^{tr/tr} mutants (FIG. 3D, FIG. 3F). Notably, the reduction was greater than the one observed in Brca1^{tr/tr}Smarcal1^{-/-} cells compared to Brca1^{tr/tr} suggesting that the HDR+SFP− genotype might have an advantage over HDR-SFP+ during reprogramming (FIG. 3D, FIG. 3F). Ablation of 53bp1 alone did not have any detectable effects on proliferation, apoptosis or development (FIG. 3G, FIG. 3H, FIG. 3I). However, loss of 53bp1 in Brca1^{tr/tr} mutants rescued their proliferation and apoptosis defects (FIG. 3G, FIG. 3H) as well as the reduced embryo size (FIG. 3I). Removal of 53bp1 in Brca1^{tr/tr} B cells, which restores HDR, also fully rescued their reprogramming defect (FIG. 3J). Additionally, 53bp1 loss consistently resulted in a significant increase in the reprogramming of HDR+SFP+ wild type and SFP− deficient Brca1^{tr/+} cells (FIG. 3J, FIG. 3M). This increase in reprogramming did not occur as consequence of impaired p21 signaling (FIG. 3O) but was due to an enhanced capacity for HDR in the absence of 53bp1 (FIG. 3K, FIG. 3L).

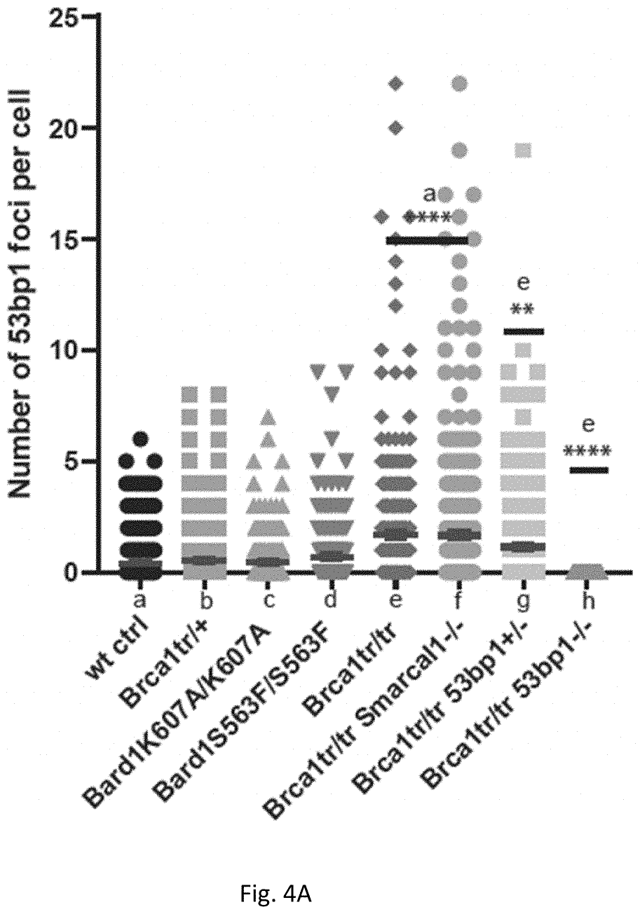

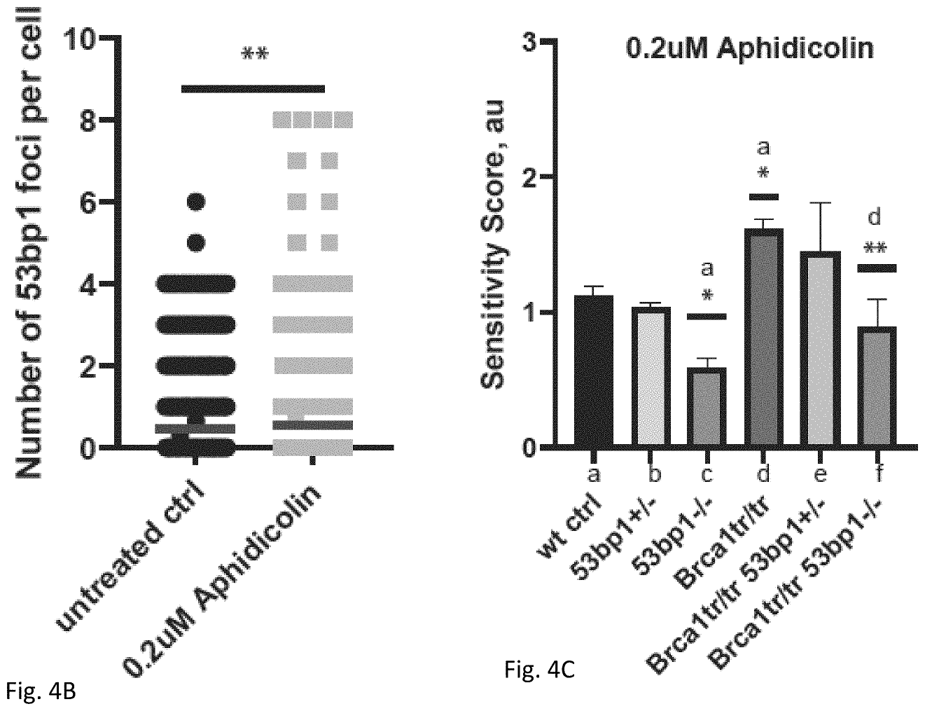

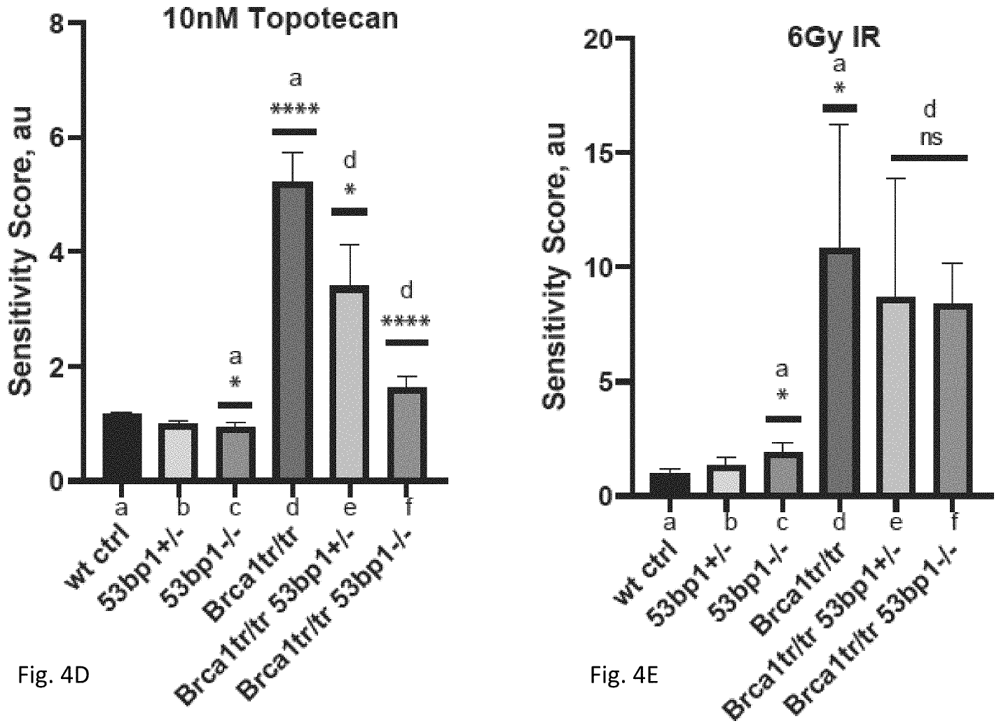

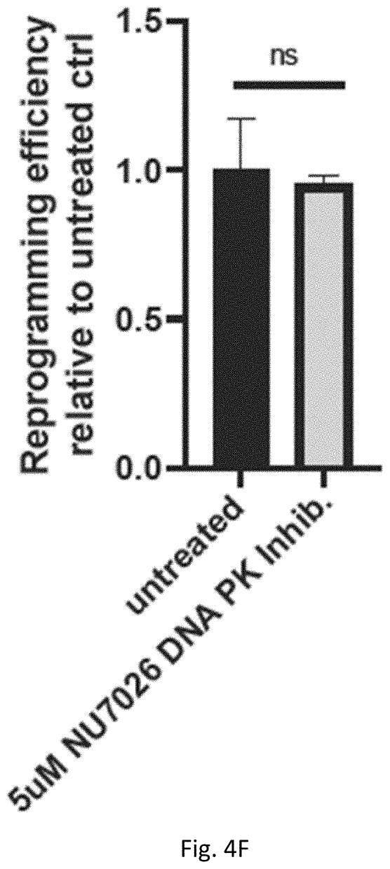

The experiments conducted thus far showed that the HDR function of BRCA1, rather than its role in SFP is required for efficient reprogramming. To investigate this further, 53bp1 focus formation in SFP− HDR+ as well as SFP− HDR− and SFP+HDR− mutant cells was looked to. All 3 SFP− deficient genotypes Brca1^{tr/+}, Bard1^{K607A/K607A} and Bard1^{S563F/S563F} formed 53bp1 foci in response to DNA damage during reprogramming at a rate similar to the one in wild-type controls (FIG. 4A). Since 53bp1 nuclear bodies typically arise by the aberrant processing of under-replicated DNA (Harrigan et al., 2011), this observation suggested that the elevated levels of DSBs that accumulate in reprogramming SFP− cells are resolved prior to the G2/M transition. In contrast, both Brca1^{tr/tr} and Brca1^{tr/tr} Smarcal1^{-/-} cells accumulated over 4-fold higher numbers of 53 bp1 foci compared to controls, demonstrating that rescue of SFP does not reduce 53bp1 focus formation as long as HDR remains compromised (FIG. 4A). The number of 53bp1 foci declined in Brca1^{tr/tr}53BP1^{+/-} fibroblasts undergoing reprogramming and no foci were detected on a 53bp1 null background (FIG. 4A). These results indicated a negative correlation between 53bp1 focus formation and reprogramming efficiency and suggested that the repair of reprogramming-induced DNA damage by 53BP1-mediated NHEJ impedes iPS cell generation.

### Example 6—One-Ended Double Strand Breaks Acquired During DNA Replication Limit Reprogramming

The DNA damage observed in the form of phosphorylated H2AX during reprogramming can be the result of global epigenetic remodeling (Hernandez et al., 2018), oxidative stress (Ji et al., 2014) or elevated replication stress (Ruiz et al., 2015). To determine the specific type of DNA damage which is most limiting to iPS cell generation, mouse embryonic fibroblasts (MEFs) were treated during reprogramming with the DNA polymerase inhibitor aphidicolin, which induces one-ended double strand breaks (DSBs), processed by HDR (Rothkamm et al., 2003). To test the effect of aphidicolin on primary cells, uninfected wild type fibroblasts were incubated with 0.2 uM aphidicolin for 3 days and noted an increase in the numbers of 53bp1 foci (FIG. 4B). The treatment of wild type cells with low concentrations of aphidicolin for 8 days during reprogramming reduced the efficiency of the process (FIG. 4C). The HDR-SFP− genotype Brca1^{tr/tr} exhibited pronounced sensitivity to aphidicolin with a significantly greater reduction in colony numbers relative to wild-type (FIG. 4C). Interestingly, 53bp1^{-/-} mutants were less sensitive to aphidicolin and there was an improvement of reprogramming efficiency of aphidicolin-treated Brca1^{tr/tr}53bp1^{-/-} cells compared to the Brca1^{tr/tr} genotype (FIG. 4C). Since aphidicolin increases the burden of one-ended DSBs during reprogramming, these results indicated that this type of damage requires repair by HDR. Cells with an increased capacity for HDR, such as 53bp1^{-/-} mutants, have reduced sensitivity to aphidicolin (FIG. 4C).

To test a different compound for the induction of one-ended double strand breaks, cells undergoing reprogramming were treated with low concentrations of topotecan, a water-soluble derivative of the topoisomerase I inhibitor camptothecin. In the presence of topotecan, DNA polymerase encounters single strand nicks, which are converted to one-ended double strand breaks during replication. While wild-type cells were barely sensitive to low doses of topotecan, the HDR− deficient genotype Brca1^{tr/tr} reprogrammed with greatly reduced efficiency (FIG. 4D). This phenotype was improved in Brca1 mutants lacking 53bp1 (Brca1^{tr/tr}53bp1^{-/-}), which experienced a mild sensitivity to topotecan, similar to the one in wild-type controls (FIG. 4D).

Two-ended double strand breaks are caused by exogenous DNA damage, endogenously by the activity of topoisomerase II or oxidative stress and can be processed by either HDR or NHEJ. To induce two-ended double strand breaks, a single administration of 6 Gy ionizing irradiation was used 1 day post reprogramming factor induction with doxycycline. In contrast to aphidicolin and topotecan, irradiation impaired the efficiency of iPS cell generation in 53bp1^{-/-} mutants to a greater extent than in wild-type controls, seen as an increased sensitivity index (FIG. 4E). The Brca1^{tr/tr} genotype was highly sensitive to the administration of IR during reprogramming, but there was no apparent reprogramming advantage of double Brca1^{tr/tr}53bp1^{-/-} mutants, unlike during treatment with aphidicolin or topotecan (FIG. 4C, FIG. 4D, FIG. 4E). Therefore, increasing the load of two-ended double strand breaks during reprograming impedes iPS cell generation not only in genotypes with compromised HDR, but also in 53bp1 mutants with less efficient NHEJ (Xu et al., 2017) as both pathways can be used to process such damage.

Since 53bp1-deficient genotypes reprogram better than controls in the absence of irradiation, two-ended DSBs are not the reprogramming-induced type of DNA damage which limits iPS cell generation. In corroboration of this observation, inhibition of DNA-PK has no effect on the efficiency of reprogramming (FIG. 4F). In contrast, cells with enhanced HDR capacity on a 53bp1 null background reprogram more efficiently in normal conditions, but also in the presence of aphidicolin or topotecan, which induce an accumulation of one-ended DSBs. These results indicate that one-ended DSBs, which require processing by HDR are the primary barrier to somatic cell reprogramming.

### Example 7—Use of a 53bp1 shRNA Increased Reprogramming Efficiency of Somatic Cells to iPS Cells

A shRNA is constructed using the nucleotide sequence of 53bp1, which is available at the National Center for Biotechnology Information Database (Gene ID: 7158). A lentiviral vector is prepared containing the shRNA and a polymerase III promoter.

A doxycycline inducible lentiviral system, consisting of Tet-O-FUW-OSKM (Addgene #20321) and FUW-M2rtTA (Addgene #20342) and the 53bp1 shRNA vector, is used. Lentivirus is prepared in 293T cells by transfection of plasmids with Jetprime transfection reagent (VWR #89129-922) as outlined in the manufacturer's instructions. Briefly, Tet-O-FUW and shRNA vectors are transfected together with the envelope and packaging plasmids from Didier Trono pMD2VSVG (Addgene #12259) and psPax2 (Addgene #12260) into 293T cells plated on collagen-coated dishes. Fresh antibiotic free media DMEM HG (Thermo Fisher Scientific #10569010), supplemented with 15% FBS (Atlanta Biologicals #S11150) and Glutamax (Thermo Fisher Scientific #35050079) is provided 16 to 20 h post transfection. Viral supernatant is collected on each of the following two days and kept at 4° C. for up to 4 days. Prior to infection, titer from the two collection days is pooled and filtered through a 40 uM cell strainer (Fisher Scientific #08-771-1).

For infection, human somatic cells are plated at 1×10⁶ cells per 10 cm dish on the previous day. Infection proceeds in two rounds with 8 h to 9 h in between. Briefly, cells are incubated with a 53bp1 shRNA/OSKM/rtta virus mix (1:1), supplemented with 8 ug/ml protamine sulfate (Fisher Scientific #0219472905). The infection mix is removed on the following day and cells are left to recover in fresh media (DMEM HG Thermo Fisher Scientific #10569010 with 10% FBS Atlanta Biologicals #S11150, Glutamax Thermo Fisher Scientific #35050079 and PenStrep Thermo Fisher Scientific 15140163).

Two days after infection, cells are re-plated for transduction efficiency assessment on day 3, molecular analyses on day 5, colony picking on day 16 and alkaline phosphatase (AP) staining on day 20. In each experiment, infected fibroblasts are replated at multiple densities to allow for optimal reprogramming efficiency. 100-300 cells/mm² (20-60K per well of a 24 w dish) routinely generated high numbers of iPS cell clones. The OSKM/shRNA reprogramming factors are induced with 1 ug/ml doxycycline (Sigma #D9891) in cell media, consisting of Knockout DMEM (Life Technologies #10829-018), supplemented with 15% Knockout Serum Replacement (Life Technologies #10828-028), Glutamax (Thermo Fisher Scientific #35050079), MEM NEAA (Life Technologies #11140050), PenStrep (Thermo Fisher Scientific 15140163), 2-mercaptoetahnol (Life Technologies #21985-023) and 10 ng/ul LIF (eBioscience #34-8521-82). Transduction efficiency is determined on reprogramming day 3 by staining for Sox2 (Stemgent #09-0024) and used in the calculation of reprograming efficiency.

The reprogramming efficiency is compared to the reprogramming efficiency of control cells reprogrammed as described above but without the use of the 53bp1 shRNA lentiviral construct. The reprogramming efficiency of the cells as well as the genome stability where the 53bp1 shRNA lentiviral construct is used is greater than that of the control cells.

### REFERENCES

Billing, et al. (2018). The BRCT Domains of the BRCA1 and BARD1 Tumor Suppressors Differentially Regulate Homology-Directed Repair and Stalled Fork Protection. Mol Cell 72, 127-139 e128.
Bunting, et al. (2010). 53BP1 inhibits homologous recombination in Brca1-deficient cells by blocking resection of DNA breaks. Cell 141, 243-254.
Canny, et al. (2018). Inhibition of 53BP1 favors homology-dependent DNA repair and increases CRISPR-Cas9 genome editing efficiency. Nature Biotechnology 136, 95-102
Cantor, et al. (2001). BACH1, a Novel Helicase-like Protein, Interacts Directly with BRCA1 and Contributes to Its DNA Repair Function. Cell 105, 149-160.
Cao, et al. (2009). A selective requirement for 53BP1 in the biological response to genomic instability induced by Brca1 deficiency. Mol Cell 35, 534-541
Chen, et al. (2018). Homology-Directed Repair and the Role of BRCA1, BRCA2, and Related Proteins in Genome Integrity and Cancer. Annu. Rev Cancer Biol 2, 313-336.
Chia, et al. (2017). Genomic instability during reprogramming by nuclear transfer is DNA replication dependent. Nat Cell Biol 19, 282-291.
Cuella-Martin, et al. (2016). 53BP1 Integrates DNA Repair and p53-Dependent Cell Fate Decisions via Distinct Mechanisms. Mol Cell 64, 51-64.
Durkin, et al. (2013). Isolation of Mouse Embryo Fibroblasts. Bio Protoc 3.
Escribano-Diaz, et al. (2013). A cell cycle-dependent regulatory circuit composed of 53BP1-RIF1 and BRCA1-CtIP controls DNA repair pathway choice. Mol Cell 49, 872-883.
Gomez-Cabello, et al. (2017). CtIP Specific Roles during Cell Reprogramming Have Long-Term Consequences in the Survival and Fitness of Induced Pluripotent Stem Cells. Stem Cell Reports 8, 432-445.
Gonzalez, et al. (2013). Homologous recombination DNA repair genes play a critical role in reprogramming to a pluripotent state. Cell Rep 3, 651-660.
Gore, et al. (2011). Somatic coding mutations in human induced pluripotent stem cells. Nature 471, 63-67.
Harrigan, et al. (2011). Replication stress induces 53BP1-containing OPT domains in G1 cells. J Cell Biol 193, 97-108.
Hernandez, et al. (2018). Dppa2/4 Facilitate Epigenetic Remodeling during Reprogramming to Pluripotency. Cell Stem Cell 23, 396-411 e398.
Hong, et al. (2009). Suppression of Induced Pluripotent Stem Cell Generation by the p53-p21 Pathway. Nature 460, 1132-1134.
Jaspers, et al. (2013). Loss of 53BP1 causes PARP inhibitor resistance in Brca1-mutated mouse mammary tumors. Cancer Discov 3, 68-81.
Ji, et al. (2014). Antioxidant supplementation reduces genomic aberrations in human induced pluripotent stem cells. Stem Cell Reports 2, 44-51.
Kareta, et al. (2015). Inhibition of pluripotency networks by the Rb tumor suppressor restricts reprogramming and tumorigenesis. Cell Stem Cell 16, 39-50.
Kawamura, et al. (2009). Linking the p53 tumour suppressor pathway to somatic cell reprogramming. Nature 460, 1140-1144.
Kinoshita, et al. (2011). Ataxia-telangiectasia mutated (ATM) deficiency decreases reprogramming efficiency and leads to genomic instability in iPS cells. Biochem Biophys Res Commun 407, 321-326.
Lezaja and Altmeyer, (2018). Inherited DNA lesions determine G1 duration in the next cell cycle. Cell Cycle 17, 24-32.
Ludwig, et al. (2001). Tumorigenesis in mice carrying a truncating Brca1 mutation. Genes Dev 15, 1188-1193.
Mao, et al. (2008). Comparison of nonhomologous end joining and homologous recombination in human cells. DNA Repair (Amst) 7, 1765-1771.
Marión, et al. (2009). A p53-mediated DNA damage response limits reprogramming to ensure iPS cell genomic integrity. Nature 460, 1149-1153.
Mason, et al. (2019). Non-enzymatic roles of human RAD51 at stalled replication forks. Nat Commun 10, 4410.
Mateos-Gomez, et al. (2017). The helicase domain of Poltheta counteracts RPA to promote alt-NHEJ. Nat Struct Mol Biol 24, 1116-1123.
Moynahan, et al. (1999). Brca1 controls homologydirected DNA repair. Mol Cell 4, 511-518.
Muller, et al. (2012). Overcoming reprogramming resistance of Fanconi anemia cells. Blood 119, 5449-5457.
Murphy, et al. (2014). Phosphorylated RPA recruits PALB2 to stalled DNA replication forks to facilitate fork recovery. J Cell Biol 206, 493-507.
Pasero and Vindigni (2017). Nucleases Acting at Stalled Forks: How to Reboot the Replication Program with a Few Shortcuts. Annu Rev Genet 51, 477-499.
Pathania, et al. (2014). BRCA1 haploinsufficiency for replication stress suppression in primary cells. Nat Commun 5, 5496.
Przetocka, et al. (2018). CtIP-Mediated Fork Protection Synergizes with BRCA1 to Suppress Genomic Instability upon DNA Replication Stress. Mol Cell 72, 568-582 e566.
Raya, et al. (2009). Diseasecorrected haematopoietic progenitors from Fanconi anaemia induced pluripotent stem cells. Nature 460, 53-59.
Rothkamm, et al. (2003). Pathways of DNA double strand break repair during the mammalian cell cycle. Mol Cell Biol 23, 5706-5715.
Ruiz, et al. (2015). Limiting replication stress during somatic cell reprogramming reduces genomic instability in induced pluripotent stem cells. Nat Commun 6, 8036.
Schlacher, et al. (2012). A distinct replication fork protection pathway connects Fanconi anemia tumor suppressors to RAD51-BRCA1/2. Cancer Cell 22, 106-116.
Schlacher, et al. (2011). Double-strand break repair-independent role for BRCA2 in blocking stalled replication fork degradation by MRE11. Cell 145, 529-542.
Shakya, et al. (2011). BRCA1 Tumor Suppression Depends on BRCT Phosphoprotein Binding, But Not Its E3 Ligase Activity. Science 334, 525-527.
Sui, et al. (2020). Establishing cell-intrinsic limitations in cell cycle progression controls graft growth and promotes differentiation of pancreatic endocrine cells.
Taglialatela, et al. (2017). Restoration of Replication Fork Stability in BRCA1- and BRCA2-Deficient Cells by Inactivation of SNF2-Family Fork Remodelers. Mol Cell 68, 414-430 e418.
Takahashi, and Yamanaka, (2006). Induction of pluripotent stem cells from mouse embryonic and adult fibroblast cultures by defined factors. Cell 126, 663-676.
Terret, et al. (2009). Cohesin acetylation speeds the replication fork. Nature 462, 231-234.
Utikal, et al. (2009). Immortalization eliminates a roadblock during cellular reprogramming into iPS cells. Nature 460, 1145-1148.
Wang, et al. (2007). Abraxas and RAP80 form a BRCA1 protein complex required for the DNA damage response. Science 316, 1194-1198.
Ward, et al. (2005). 53BP1 cooperates with p53 and functions as a haploinsufficient tumor suppressor in mice. Mol Cell Biol 25, 10079-10086.
Wu, et al. (1996). Identification of a RING protein that can interact in vivo with the BRCA1 gene product. Nature Genetics 14, 430-440.
Xu, et al. (2017). 53BP1 and BRCA1 control pathway choice for stalled replication restart. Elife 6.
Yu, et al. (1998). The C-terminal (BRCT) Domains of BRCA1 Interact in Vivo with CtIP, a Protein Implicated in the CtBP Pathway of Transcriptional Repression. The Journal of Biological Chemistry 273, 25388-25392.

1. A method of generating a human induced pluripotent stem (iPS) cell, comprising: (a) introducing an agent which inhibits, reduces, knock downs or down regulates 53BP1 expression into an isolated human somatic cell; and (b) culturing the cell obtained in step a) under conditions to generate a human induced pluripotent stem cell.

2. The method of claim 1, wherein the somatic cell is an epidermal cell, fibroblast, blood cell, mammary epithelial cell, lung epithelial cell, or intestinal epithelial cell.

3. The method of claim 1, wherein the somatic cell is allogeneic or autologous.

4. The method of claim 1, wherein the agent which inhibits, reduces, knock downs or down regulates 53BP1 is an inhibitory nucleic acid.

5. The method of claim 4, wherein the inhibitory nucleic acid is shRNA, siRNA, or miRNA.

6. The method of claim 4, wherein the inhibitory nucleic acid is present in an expression vector.

7. The method of claim 6, wherein the expression vector is chosen from the group consisting of a lentiviral vector, a retroviral vector, an adenoviral vector, an episomal vector or a plasmid.

8. The method of claim 4, wherein the inhibitory nucleic acid is present on an expression vector further comprising four transcription factors OCT4, SOX2, KLF4 and cMYC (OSKM).

9. The method of claim 1, wherein the agent is a polypeptide or protein.

10. The method of claim 1, wherein the agent is an endonuclease.

11. The method of claim 10, wherein the endonuclease is chosen from the group consisting of a zinc finger nuclease (ZFN), a ZFN dimer, a ZFNickase, a transcription activator-like effector nuclease (TALEN), and a RNA-guided DNA endonuclease (CRISPR/Cas9).

12. The method of claim 1, wherein the agent is introduced into the somatic cell at the same time as four transcription factors OCT4, SOX2, KLF4 and cMYC (OSKM).

13. The method of claim 1, further comprising the step of subjecting the induced pluripotent stem cell to conditions to produce a differentiated cell.

14. The method of claim 1, wherein 53BP1 expression is regained in the human induced pluripotent stem cell.

15. An expression vector comprising a nucleic acid which inhibits, reduces, knock downs or down regulates 53BP1 expression, for use in the method of claim 1.

16. The expression vector of claim 14, wherein the nucleic acid is chosen from the group consisting of shRNA, siRNA, or miRNA.

17. The expression vector of claim 14, wherein the vector is chosen from the group consisting of a lentiviral vector, a retroviral vector, an adenoviral vector, an episomal vector or a plasmid.

18. A kit comprising the expression vector of claim 15.

19. The kit of claim 18, further comprising: a somatic cell; a vector comprising four transcription factors OCT4, SOX2, KLF4 and cMYC (OSKM); and media.


SEQUENCE LISTING (19 sequences)

SEQ ID NO: 1 - DNA, 20 bp, Artificial Sequence
  Synthetic Primer
gcaggtgctctaccctcaac

SEQ ID NO: 2 - DNA, 18 bp, Artificial Sequence
  Synthetic Primer
aacctggccatcaacatg

SEQ ID NO: 3 - DNA, 20 bp, Artificial Sequence
  Synthetic Primer
cacgtggttgctggaaattg

SEQ ID NO: 4 - DNA, 19 bp, Artificial Sequence
  Synthetic Primer
atgtaaaggagccagcagc

SEQ ID NO: 5 - DNA, 20 bp, Artificial Sequence
  Synthetic Primer
tgctcactctgtgccctcaa

SEQ ID NO: 6 - DNA, 20 bp, Artificial Sequence
  Synthetic Primer
tccattctccccgcttctgt

SEQ ID NO: 7 - DNA, 20 bp, Artificial Sequence
  Synthetic Primer
ccgctctaacctgggaacac

SEQ ID NO: 8 - DNA, 20 bp, Artificial Sequence
  Synthetic Primer
gtgacagacaacagccagcc

SEQ ID NO: 9 - DNA, 20 bp, Artificial Sequence
  Synthetic Primer
tcgtggtatcgttatgcgcc

SEQ ID NO: 10 - DNA, 21 bp, Artificial Sequence
  Synthetic Primer
aggagactgaagaaccaatcg

SEQ ID NO: 11 - DNA, 21 bp, Artificial Sequence
  Synthetic Primer
ctcagttttcctgggcctcct

SEQ ID NO: 12 - DNA, 20 bp, Artificial Sequence
  Synthetic Primer
gtcagggtttcactggcttg

SEQ ID NO: 13 - DNA, 18 bp, Artificial Sequence
  Synthetic Primer
ccttcttgacgagttctt

SEQ ID NO: 14 - DNA, 20 bp, Artificial Sequence
  Synthetic Primer
cagcaggcaccaagacaagg

SEQ ID NO: 15 - DNA, 27 bp, Artificial Sequence
  Synthetic Primer
tctgtgtattaatccgagaggcaaaga

SEQ ID NO: 16 - DNA, 20 bp, Artificial Sequence
  Synthetic Primer
gccaagtgtcccagctcaaa

SEQ ID NO: 17 - DNA, 21 bp, Artificial Sequence
  Synthetic Primer
tcagtgtcccaggcaactaag

SEQ ID NO: 18 - DNA, 21 bp, Artificial Sequence
  Synthetic Primer
tagcaaaagtcctcagtgggc

SEQ ID NO: 19 - DNA, 20 bp, Artificial Sequence
  Synthetic Primer
tgttgctaaagggagctgtc